(19) United States
(12) Patent Application Publication
(10) Pub. No.: US 2008/0305054 A1
(43) Pub. Date: Dec. 11, 2008

(54) Use of Glycosylated Flavanones for the Browning of Skin or Hair

(75) Inventors: Gabriele Vielhaber, Holzminden (DE); Karin Schaper, Linnenkamp (DE); Martina Herrmann, Hameln (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/577,846

(22) PCT Filed: Oct. 21, 2005
(86) PCT No.: PCT/EP05/55464

§ 371 Date: Mar. 10, 2008

Related U.S. Application Data
(60) Provisional application No. 60/621,855, filed on Oct. 25, 2004.

(51) Int. Cl. A61K 8/49; A61Q 5/10; A61Q 17/04

(52) U.S. Cl. 424/59; 8/405

(57) ABSTRACT

The use of a compound having formula (I) is described as an agent for the browning of skin or hair, Formula (I) wherein: R1 and R2 are mutually independently H, OH, C1-C10-alkyl, C1-C10-O-alkyl or O-prenyl, R3 is H, OH, O-glucose or O-rhamnose and R4 is a monosaccharide radical or an oligosaccharide radical having 2, 3, 4 or 5 carbohydrate units, with the proviso that the compound having formula (I) is not used in the form of a preparation based on Citrus auranitium dulcis.

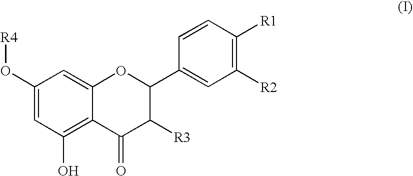

The invention concerns the use of specific glycosylated flavanones as agents for the browning of skin and/or hair in vivo.

Skin-browning active ingredients intervene in one form or another in melanin metabolism or catabolism. The melanin pigments, which are normally brown to black in colour, are formed in the melanocytes of the skin, transferred to the keratinocytes and give the skin or hair its colour. In mammals, the brown-black eumelanins are primarily formed from hydroxy-substituted aromatic amino acids such as L-tyrosine and L-3,4-dihydroxyphenyl alanine (L-DOPA), which additionally forms the yellow to red pheomelanins from sulfur-containing molecules (Cosmetics & Toiletries 1996, 111 (5), 43-51). Starting from L-tyrosine, L-DOPA is formed by the copper-containing key enzyme tyrosinase and is in turn converted by tyrosinase to dopachrome. By a series of steps catalysed by various enzymes, the latter is oxidised to form melanin.

In the presence of UV radiation the melanocytes increasingly form melanin. On the one hand this acts as natural UV protection. On the other, melanin is an antioxidant, which protects against reactive oxygen species (oxidative stress).

UV radiation has many damaging side-effects:

UV-B radiation (290 nm and 320 nm) can lead to the formation of erythema or even to burns.

UV-A radiation (320-400 nm) can cause skin damage by damaging the keratin or elastin in the skin. This reduces the elasticity and water-retaining ability of the skin, in other words the skin becomes less supple and has a tendency to form wrinkles. The remarkably high incidence of skin cancer in areas of high solar radiation shows that sunlight evidently also damages the genetic information in the cells.

UV radiation can also lead to photochemical reactions, wherein the photochemical reaction products—such as e.g. hydroxyl radicals or singlet oxygen—interfere with the metabolism of the skin. Undefined radical photoproducts occurring in the skin itself can also lead to uncontrolled secondary reactions due to their high reactivity.

In addition, UV radiation is classed as ionising radiation. There is therefore a risk of the formation of ionic species under UV exposure, which in turn can then influence the biochemical processes by oxidation.

Skin-browning agents are used for various reasons:

If for some reason the melanin-forming melanocytes in human skin are not evenly distributed, pigment spots occur which are either lighter or darker than the surrounding skin area. To overcome this problem, skin and hair browning agents are sold which at least partially help to balance out such pigment spots. In addition, many people need to tint their naturally pale skin colour and to develop skin pigmentation without being exposed to solar radiation. For this reason very safe and effective skin and hair browning agents are necessary.

It is also known that in fair-skinned people high exposure to the sun can cause the breakdown of the vitally important B vitamin folic acid. Folic acid deficiency in pregnancy for example leads to severe deformities. Folic acid is also necessary for DNA synthesis and is therefore essential for sperm production. Folic acid deficiency can therefore lead to infertility. A protection against UV radiation accordingly prevents folic acid deficiency.

Artificial skin browning can be carried out cosmetically or medically, wherein the following approaches play a part:

If carotene preparations are taken regularly, carotene is stored in the fatty tissue of the subcutis and the skin gradually turns orange to yellow-brown.

Washable makeup preparations can be used to achieve a light skin tinting (e.g. extracts of fresh green walnut shells, henna).

Skin browning can also be achieved by chemical changes to the skin's stratum corneum using so-called self-tanning preparations. The most important active ingredient is dihydroxyacetone (DHA). The skin browning achieved in this way does not wash off and is only removed with the normal flaking of the skin (after around 10 to 15 days). Dihydroxyacetone can be classed as a ketotriose and as a reducing sugar it reacts with the amino acids in the skin or the free amino and imino groups in keratin via a series of intermediate steps along the lines of a Maillard reaction to form brown-coloured substances known as melanoids, which are occasionally also called melanoidins.

One disadvantage of this is that unlike “sun-tanned” skin, the skin browning obtained with dihydroxyacetone does not protect the skin against sunburn. A further disadvantage of dihydroxyacetone lies in the fact that, particularly under the influence of ultraviolet radiation, it releases formaldehyde, albeit usually in small amounts.

The tint obtained with self-tanning agents is achieved without exposure to sunlight. In contrast, so-called “pre-tan products” or “tan promoters” are also available, which have to be applied before exposure to sunlight. In the sun these products then turn yellow, giving rise to a light brown-yellow colouring of the epidermis which further boosts the “suntan”.

Another type of artificial browning which is not dependent on UV light can be brought about through the hormones which are usually also released in the body as a consequence of (natural) UV irradiation and ultimately stimulate the melanocytes to synthesise melanin. Examples which can be cited in this connection are derivatives of proopiomelanocortin (POMC) such as α-MSH (Melanocyte Stimulating Hormone) and synthetic variants (such as [Nle(4), D-Phe(7)]-α-MSH), which in some cases display far higher activity levels than the natural α-MSH. Although these hormones can cause browning in principle, their use in cosmetics is prohibited, since they are pharmacologically potent substances (hormones) which should not be widely used without medical indications.

The use of tyrosinase substrates such as L-tyrosine, L-DOPA and derivatives or precursors thereof for the stimulation of melanogenesis has also been described many times in the literature.

The topical use of flavonoids to accelerate skin melanogenesis is described—in very general terms—in JP 2004002264. The flavonoids in this case include structures having the following general structural formulae:

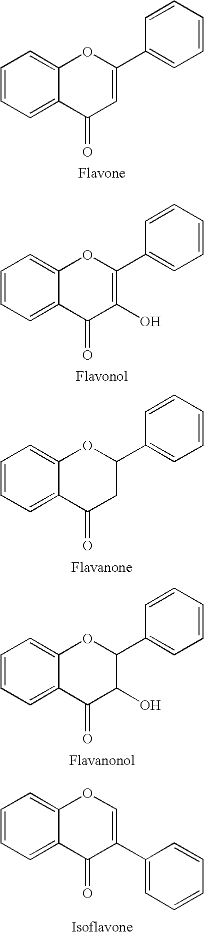

Flavonoids having structures in accordance with the above structural formulae do not have a uniform effect on melanogenesis, however:
Badria and el Gayyar (Boll Chim Farm 2001, 140, 267-271): of 27 flavonoids tested (flavanols, flavonols, flavones, flavanones, isoflavones, aflavins, anthocyanidins) the flavonols quercetin, myricetin, morin and kaempferol and the aflavins theaflavin, theaflavinmonogallate and the flavone luteolin displayed a tyrosinase inhibition. Tyrosinase inhibition generally has a skin-lightening effect.
Wang et al. (J Nutr Biochem 2002, 13, 421-427) found that genistein (an isoflavone) increases melanin production in melanoma cells, whilst daidzein (an isoflavone) has no effect on melanogenesis. (Note: Isoflavones disadvantageously have oestrogenic effects (Breinholt et al. Food Chem Toxicol, 2000, 38(7): 555-64).
Drewa et al. (Neoplasma 1998, 45, 266-271) showed that rutin (quercetin 3-β-D-rutinoside) inhibits melanin formation in B16 cells.
Serafino et al. (FASEB J published online Sep. 27, 2004) showed that the anthocyanidin cyanidin-3-β-D-glucoside stimulates melanin synthesis in melanoma cells. Cyanidins have a strong red coloration, however, and are therefore unsuitable for use in cosmetics.
Kim et al (Arch Pharm Res 2004, 27, 334-339) describe an inhibition of melanin synthesis by the flavanol epigallocatechin gallate.

According to JP 2004002264 the use of the flavonols quercetin, rhamnetin, kaempferol and fisetin, the isoflavones genistein and daidzein and the flavones chrysin and apigenin is preferred. The most effective flavonoids were luteolin and quercetin. As our investigations showed, however, these melanogenesis stimulators are only effective in cytotoxic concentrations. In addition, quercetin was shown to have a hepatotoxic effect in male rats and a mutagenic effect (Natl Toxicol Program Tech Rep Ser409, 1-171).

Shoji et al. (Bioscience, Biotechnology, and Biochemistry 1997, 61 (12), 1963-1937) describe the stimulating action of polyphenols and flavonoids on the melanogenesis of B16V melanoma cells, among them quercetin, kaempferol and the dihydrochalcones phloridzin and phloretin. Phloridzin (R=glucose) and phloretin (R═H) have the following structure:

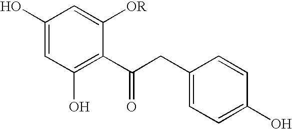

All compounds were investigated in non-cytotoxic concentrations. Phloridzin had the strongest effect (1 mM=+81% melanin relative to the untreated control) and displayed no cytotoxicity, whilst the aglycone phloretin was ineffective. However, phloridzin acts as an inhibitor of glucose absorption and is therefore toxic to reproduction (Leppens-Luisier et al. 2001, Human Reproduction 16(6), 1229-1236). Of the other flavonoids, quercetin was the most effective, but in the maximum non-cytotoxic concentration of 0.003 mM with +38% melanin it displayed only a moderate effect.

FR 2845285 describes the use of a preparation containing flavonoid polyphenols based on Citrus aurantium dulcis powder for skin browning.

The object from a cosmetic perspective is therefore to provide alternative, effective skin and hair browning compounds which are non-toxic and are well tolerated in effective amounts.

The object is also to provide alternative compounds to stimulate the natural melanogenesis of human skin, especially in connection with the prevention of folic acid deficiency phenomena and the treatment of pigment spots.

According to a first aspect of the present invention the stated objects are achieved by the use of a compound having formula (I) as an agent for the browning of skin or hair in vivo,

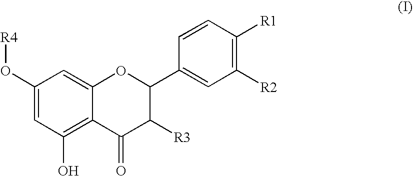

wherein:
R1 and R2 are mutually independently H, OH, C1-C10-alkyl, C1-C10-O-alkyl or O-prenyl,

### R3 is H, OH, O-glucose or O-rhamnose

and
R4 is a monosaccharide radical or an oligosaccharide radical having 2, 3, 4 or 5 carbohydrate units,
with the proviso that the compound having formula (I) is not used in the form of a preparation based on Citrus aurantium dulcis.

The compound having formula (I) is preferably used in an amount which is in the range between the minimum necessary amount for browning in vivo and 1000 times, preferably 100 times, this amount. Such an amount generally excludes a cytotoxic effect; cf. in this connection examples 12 to 14 below which, although they relate to in-vitro experiments, contain results which are probably transferable in this respect.

The compounds having formula (I), glycosylated flavanones, include their stereoisomers and anomers and any mixtures of these isomers.

Compounds having formula (I), their stereoisomers and anomers have surprisingly proved to be particularly effective stimulators of the natural melanogenesis of human skin and can be readily processed with the constituents of common cosmetic formulations. This is expressly stated once more at this point.

Compounds having formula (I) are preferably used in which:

R1 and R2 are mutually independently H, OH, OMe or CH₃
and

### R3 is H

and
R4 is a monosaccharide radical or an oligosaccharide radical having 2, 3, 4 or 5 carbohydrate units.

The use of compounds having formula (I) is particularly preferred in which:

R1 and R2 are mutually independently H, OH or OMe
and

### R3 is H

and
R4 is (i) a monosaccharide radical, preferably selected from the group consisting of glucose, galactose, rhamnose, xylose and glucuronic acid, or (ii) a disaccharide radical, whose sugar units are the same or different and are preferably selected from the group consisting of glucose, galactose, rhamnose, xylose and glucuronic acid.

The use of the following compounds having formula (I), which have a particularly good ratio of minimum effective amount to minimum cytotoxic amount, has proved to be particularly advantageous:

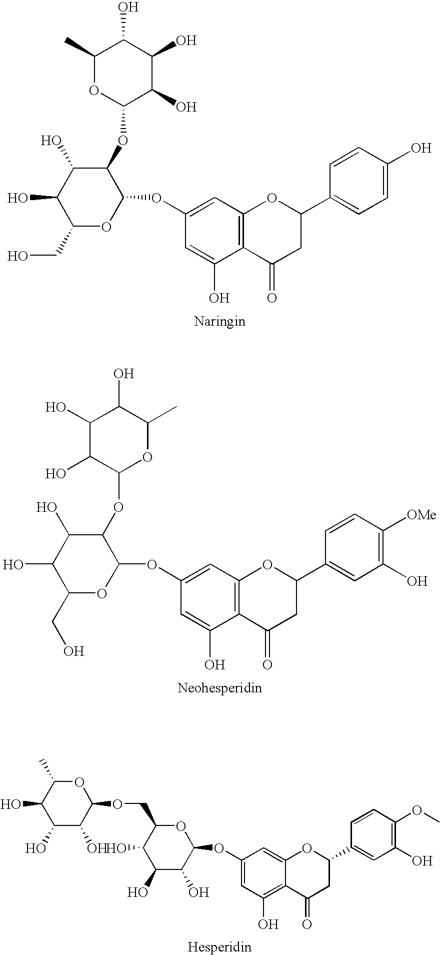

Naringin is particularly preferred, cf. also the examples in this connection.

The invention also concerns a corresponding process for the browning of skin or hair, with the following step:
Application of an effective amount of a compound having formula (I) in vivo to the hair or skin.

Preferred embodiments of the process according to the invention correspond to preferred embodiments of the use and formulation according to the invention.

The compounds having formula (I) for use according to the invention strengthen the pigmentation of melanocytes, i.e. they stimulate the natural melanogenesis of human skin. This effect is independent of the presence of UV light but can be strengthened by UV light. They can therefore be used as active ingredients in cosmetic or therapeutic, in particular dermatological, skin and hair browning agents. The compounds having formula (I) are also potent and non-cytotoxic in the concentrations in which they are used.

The present invention accordingly also concerns a formulation (particularly a topical cosmetic or therapeutic formulation), in particular a cosmetic (optionally dermatological) skin and hair browning agent, for the browning of skin or hair, comprising
at least one compound having formula (I),

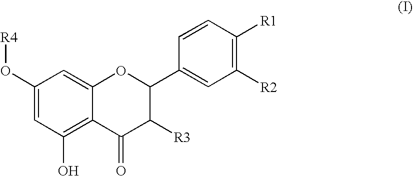

wherein:
R1 and R2 are mutually independently H, OH, C1-C10-alkyl, C1-C10-O-alkyl or O-prenyl,

### R3 is H, OH, O-glucose or O-rhamnose

and
R4 is a monosaccharide radical or an oligosaccharide radical having 2, 3, 4 or 5 carbohydrate units,
in an amount which is in the range between the minimum necessary amount for browning in vivo and 1000 times, preferably 100 times, that amount,
with the proviso that the compound having formula (I) is not used in the form of a preparation based on Citrus aurantium dulcis,
a skin or hair conditioning or cleansing substance and
optionally one or more (conventional) additives.

The (cosmetic or therapeutic) formulations according to the invention are produced by conventional processes known per se, such that one or more of the glycosyl flavanones having formula (I) used according to the invention are incorporated into cosmetic or dermatological formulations which have a conventional composition and which in addition to the skin and hair browning effect can also be used for the treatment, care and cleansing of the skin or hair and as makeup products in decorative cosmetics.

Formulations according to the invention preferably contain 0.01 wt. % to 30 wt. %, preferably 0.01 to 20 wt. %, but in particular 0.05 wt. % to 5 wt. % and preferably 0.1 to 1 wt. %, relative to the total weight of the formulation, of the glycosyl flavanones having formula (I) for use according to the invention and can take the form for example of soap, synthetic detergent, liquid washing, shower and bath preparation, emulsion (as a solution, dispersion, suspension; cream, lotion or milk depending on the production process and ingredients as a W/O, O/W or multiple emulsion, PIT emulsion, emulsion foam, micro-emulsion, nano-emulsion, Pickering emulsion), as an ointment, paste, gel (including hydrogel, hydrodispersion gel, oleogel), oil, toner, balsam, serum, powder, eau de toilette, toilette, eau de cologne, perfume, wax, as a stick, roll-on, (pump) spray, aerosol (foaming, non-foaming or post-foaming), as a foot care product (including keratolytics, deodorants), as a shaving foam or aftershave (balm, lotion) as a depilatory product, hair care product such as e.g. shampoo (including 2-in-1 shampoo), conditioner, hair tonic, hair water, hair rinse, hair cream, pomade, perm and setting lotion, hair smoothing product (detangling product, relaxer), hair strengthener, styling aid (e.g. gel or wax); blonding product, hair dye (e.g. temporary hair dyes, colour rinses, semi-permanent and permanent hair dyes), as nail care products such as e.g. nail polish and nail polish remover, as deodorants and/or antiperspirants; mouthwash, makeup, makeup remover, decorative cosmetics (e.g. powder, eyeshadows, kohl pencil, lipstick).

It is also advantageous to administer the glycosol flavanones in encapsulated form, e.g. in gelatine, wax materials, liposomes, cellulose or cyclodextrin capsules. Other conventional cosmetic auxiliary substances and additives can be included in quantities of 5 to 99 wt. %, preferably 10 to 80 wt. %, relative to the total weight of the formulation. The formulations can also have water in a quantity of up to 99.99 wt. %, preferably 5 to 80 wt. %, relative to the total weight of the formulation.

There are no references to a pigmenting effect, in particular a stimulating effect on the natural melanogenesis of human skin, of compounds having formula (I) or to their use in skin and hair browning agents in the prior art. Furthermore, the use of the compounds having formula (I) to prevent folic acid deficiency phenomena has hitherto been unknown.

Compounds having formula (I) are available commercially. They can however also be obtained by extraction from plants, from plants of the Rutaceae family, in particular of the Citrus species, from plants of the Rosaceae family, in particular of the Prunus species, from Ceterach officinarum, Origanum vulgare (oregano), Adiantum spp., Clymenia polyandra, from plants of the species Mentha, Vernonia, Anthurium, Xanthoxylum spp., Agathosma betulina (honeybush), Barosma betulina, Hyssopus officinalis, from plants of the Coniferae, Erythroxylaceae, Solanaceae, Hydrangaceae, Compositae, Salicaceae, Cruciferae, Filicaceae, Corariaceae, Cochlospermaceae, Leguminosae and Scrophulariaceae families and from Camellia sinensis (tea).

The cosmetic or therapeutic (especially topical) formulations according to the invention, in particular skin and hair browning agents, can contain cosmetic auxiliary substances and additives such as are conventionally used in such preparations, e.g. sunscreens, preservatives, bactericides, fungicides, virucides, cooling agents, insect repellents (e.g. DEET, IR 3225, Dragorepel), plant extracts, anti-inflammatory agents, substances to accelerate wound healing (e.g. chitin or chitosan and derivatives thereof), film-forming substances (e.g. polyvinyl pyrrolidones or chitosan or derivatives thereof), conventional anti-oxidants, vitamins (e.g. vitamin C and derivatives, tocopherols and derivatives, vitamin A and derivatives), 2-hydroxycarboxylic acids (e.g. citric acid, malic acid, L-, D- or di-lactic acid), skin colouring agents (e.g. walnut extracts or dihydroxyacetone), agents to promote hair growth (e.g minoxidil, diphencyprone, hormones, finasteride, phytosterols such as e.g. β-sitosterol, biotin or extracts of Cimicifuga racemosa, Eugenia caryophyllata or Hibiscus rosasinensis, barley, hops, hydrolysates of rice or wheat), skin conditioning agents (e.g. cholesterol, ceramides, pseudoceramides), softening, moisturising or moisture-retaining substances (e.g. glycerol or urea), fats, oils, saturated fatty acids, monounsaturated or polyunsaturated fatty acids, α-hydroxy acids, polyhydroxy fatty acids or derivatives thereof (e.g. linoleic acid, α-linolenic acid, γ-linolenic acid or arachidonic acid and the natural or synthetic esters thereof), waxes or other conventional constituents of a cosmetic or dermatological formulation such as alcohols, polyols, polymers, foam stabilisers, electrolytes, organic solvents, silicone derivatives or chelating agents (e.g. ethylene diamine tetraacetic acid and derivatives), anti-dandruff agents (e.g. climbazole, ketoconazole, piroctone oleamine, zinc pyrithione), hair conditioning agents, perfumes, substances to prevent foaming, dyes, pigments having a colouring action, thickeners (advantageously silicon dioxide, aluminium silicates, such as e.g. bentonites, polysaccharides or derivatives thereof, e.g. hyaluric acid, guar gum, xanthan gum, hydroxypropyl methylcellulose or allulose derivatives, particularly advantageously polyacrylates such as e.g. carbopols or polyurethanes), surface-active substances, emulsifiers, plant parts and plant extracts (e.g. arnica, aloe, beard lichen, ivy, stinging nettle, ginseng, henna, camomile, marigold, rosemary, sage, horsetail or thyme), animal extracts such as e.g. royal jelly, propolis, proteins, protein hydrolysates, yeast extracts, hop and wheat extracts, peptides or thymus extracts.

The amounts of cosmetic (optionally dermatological) auxiliary agents and additives and perfume to be used in each case can easily be determined by the person skilled in the art by trial and error, depending on the nature of the particular product.

The formulations according to the invention can preferably also contain other active ingredients which stimulate skin and hair tinting or browning by chemical or natural means. A more rapid action based on synergistic effects is achieved in this way. Particularly preferred here are substrates or substrate analogues of tyrosinase such as L-tyrosine, L-DOPA or L-dihydroxyphenylalanine, stimulators of tyrosinase activity or expression such as theophylline, caffeine, proopiomelanocortin peptides such as ACTH, alpha-MSH, peptide analogues thereof and other substances which bind to the melanocortin receptor, peptides such as Val-Gly-Val-Ala-Pro-Gly, Lys-Ile-Gly-Arg-Lys or Leu-Ile-Gly-Lys, purines, pyrimidines, folic acid, copper salts such as copper gluconate, chloride or pyrrolidonate, 1,3,4-oxadiazole-2-thiols such as 5-pyrazin-2-yl-1,3,4-oxadiazole-2-thiol, curcumin, zinc diglycinate (Zn(Gly)₂), manganese(II) bicarbonate complexes (“pseudocatalases”) as described for example in EP 0584178, tetrasubstituted cyclohexene derivatives as described for example in WO 2005032501, melanin derivatives such as Melasyn-100 and MelanZe, diacyl glycerols, aliphatic or cyclic diols, psoralens, prostaglandins and analogues thereof, activators of adenylate cyclase and compounds which activate the transfer of melanosomes into keratinocytes such as serine proteases or agonists of the PAR-2 receptor, extracts of plants and plant parts of the chrysanthemum species, sanguisorba species, walnut extracts, urucum extracts, rhubarb extracts, erythrulose and dihydroxyacetone.

The preparations according to the invention advantageously contain at least one UVA filter and/or at least one UVB filter and/or at least one inorganic pigment. The preparations can be in various forms, such as are conventionally used for example for sunscreen preparations to protect the skin and hair against ultraviolet radiation. Thus for example they can form a solution, a water-in-oil (W/O) or oil-in-water (O/W) emulsion, or a multiple emulsion, of the water-in-oil-in-water (W/O/W) type for example, a gel, a hydrodispersion, a solid stick or an aerosol. The total amount of filter substances here is 0.01 wt. % to 40 wt. %, preferably 0.1% to 10 wt. %, in particular 1.0 to 5.0 wt. %, relative to the total weight of the preparations, to provide cosmetic preparations.

Advantageous UV filters are, for example:
p-aminobenzoic acid
p-aminobenzoic acid ethyl ester (25 mol) ethoxylated
p-dimethylaminobenzoic acid-2-ethylhexyl ester
p-aminobenzoic acid ethyl ester (2 mol) N-propoxylated
p-aminobenzoic acid glycerol ester
salicylic acid homomethyl ester (homosalates) (Neo Heliopan®HMS)
salicylic acid-2-ethylhexyl ester (Neo Heliopan®OS)
triethanolamine salicylate
4-isopropyl benzyl salicylate
anthranilic acid menthyl ester (Neo Heliopan®MA)
diisopropyl cinnamic acid ethyl ester
p-methoxycinnamic acid-2-ethylhexyl ester (Neo Heliopan®AV)
diisopropyl cinnamic acid methyl ester
p-methoxycinnamic acid isoamyl ester (Neo Heliopan®E 1000)
p-methoxycinnamic acid diethanolamine salt
p-methoxycinnamic acid isopropyl ester
2-ethylhexyl-2-cyano-3,3-diphenyl acrylate (Neo Heliopan®303)
ethyl-2-cyano-3,3′-diphenyl acrylate
2-phenylbenzimidazole sulfonic acid and salts (Neo Heliopan®Hydro)
3-(4′-trimethylammonium) benzylidene bornan-2-one methyl sulfate
terephthalylidene dibornane sulfonic acid and salts (Mexoryl®SX)
4-t-butyl-4′-methoxydibenzoyl methane (avobenzone)/(Neo Heliopan®357)
β-imidazole-4(5)-acrylic acid (urocanic acid)
2-hydroxy-4-methoxybenzophenone (Neo Heliopan®BB)
2-hydroxy-4-methoxybenzophenone-5-sulfonic acid
dihydroxy-4-methoxybenzophenone
2,4-dihydroxybenzophenone
tetrahydroxybenzophenone
2,2′-dihydroxy-4,4′-dimethoxybenzophenone
2-hydroxy-4-n-octoxybenzophenone
2-hydroxy-4-methoxy-4′-methyl benzophenone
3-(4′-sulfo)benzylidene bornan-2-one and salts
3-(4′-methyl benzylidene)-d,l-camphor (Neo Heliopan®MBC)
3-benzylidene-d,l-camphor
4-isopropyl dibenzoyl methane
2,4,6-trianilino-(p-carbo-2′-ethylhexyl-1′-oxy)-1,3,5-triazine
phenylene bis-benzimidazyl tetrasulfonic acid disodium salt (Neo Heliopan®AP)
2,2′-(1,4-phenylene)-bis-(1H-benzimidazole-4,6-disulfonic acid), monosodium salt
N-[(2 and 4)-[2-(oxoborn-3-ylidene)methyl]benzyl]acrylamide polymer
phenol,-(2H-benzotriazol-2-yl)-4-methyl-6-(2-methyl-3(1,3,3,3-tetramethyl-1-(trimethylsilyl)oxy)disiloxyanyl)propyl) (Mexoryl®XL)
4,4′-[(6-[4-(1,1-dimethyl)aminocarbonyl)phenylamino]-1,3,5-triazine-2,4-diyl)diimino]-bis-(benzoic acid-2-ethyl hexyl ester) (Uvasorb®HEB)
2,2′-methylene bis-(6-(2H-benzotriazol-2-yl)-4-1,1,3,3-tetramethylbutyl)phenol) (Tinosorb®M)
2,4-bis-[4-(2-ethylhexyloxy)-2-hydroxyphenyl]-1,3,5-triazine
benzylidene malonate polysiloxane (Parsol®SLX)
glyceryl ethylhexanoate dimethoxycinnamate
disodium-2,2′-dihydroxy-4,4′-dimethoxy-5,5′-disulfobenzophenone
dipropylene glycol salicylate
sodium hydroxymethoxybenzophenone sulfonate
4,4′,4-(1,3,5-triazine-2,4,6-triyltriimino)-tris-benzoic acid tris(2-ethylhexyl ester) (Uvinul®T150)
2,4-bis-[{(4-(2-ethyl hexyloxy)-2-hydroxy}phenyl]-6-(4-methoxyphenyl)-1,3,5-triazine (Tinosorb®S)
2,4-bis-[{(4-(3-sulfonato)-2-hydroxypropyloxy)-2-hydroxy}phenyl]-6-(4-methoxyphenyl)-1,3,5-triazine sodium salt
2,4-bis-[{(3-(2-propyloxy)-2-hydroxypropyloxy)-2-hydroxy}phenyl]-6-(4-methoxyphenyl)-1,3,5-triazine
2,4-bis-[{4-(2-ethylhexyloxy)-2-hydroxy}phenyl]-6-[4-(2-methoxyethyl carbonyl)phenylamino]-1,3,5-triazine
2,4-bis-[{4-(3-(2-propyloxy)-2-hydroxypropyloxy)-2-hydroxy}phenyl]-6-[4-(2-ethylcarboxyl)phenylamino]-1,3,5-triazine
2,4-bis-[{4-(2-ethylhexyloxy)-2-hydroxy}phenyl]-6-(1-methylpyrrol-2-yl)-1,3,5-triazine
2,4-bis-[{4-tris-(trimethylsiloxysilylpropyloxy)-2-hydroxy}phenyl]-6-(4-methoxyphenyl)-1,3,5-triazine
2,4-bis-[{4-(2″-methylpropenyloxy)-2-hydroxy}phenyl]-6-(4-methoxyphenyl)-1,3,5-triazine
2,4-bis-[{4-(1′,1′,1′,3′5′, 5′,5′-heptamethylsiloxy-2″-methylpropyloxy)-2-hydroxy}phenyl]-6-(4-methoxyphenyl)-1,3,5-triazine
2-(4-diethylamino-2-hydroxybenzoyl)benzoic acid hexyl ester (Uvinul® A Plus)
indanylidene compounds in accordance with DE 100 55 940 (=WO 02/38537)

UV absorbers which are particularly suitable for combining are
p-aminobenzoic acid
3-(4′-trimethylammonium) benzylidene bornan-2-one methyl sulfate
salicylic acid homomethyl ester (Neo Heliopan®HMS)
2-hydroxy-4-methoxybenzophenone (Neo Heliopan®BB)
2-phenylbenzimidazole sulfonic acid (Neo Heliopan®Hydro)
terephthalylidene dibornane sulfonic acid and salts (Mexoryl®SX)
4-tert-butyl-4′-methoxydibenzoyl methane (Neo Heliopan®357)
3-(4′-sulfo)benzylidene bornan-2-one and salts
2-ethylhexyl-2-cyano-3,3-diphenyl acrylate (Neo Heliopan®303)
N-[(2 and 4)-[2-(oxoborn-3-ylidene) methyl]benzyl]acrylamide polymer
p-methoxycinnamic acid-2-ethylhexyl ester (Neo Heliopan®AV)
p-aminobenzoic acid ethyl ester (25 mol) ethoxylated
p-methoxycinnamic acid isoamyl ester (Neo Heliopan®E1000)
2,4,6-trianilino-(p-carbo-2′-ethylhexyl-1′-oxy)-1,3,5-triazine (Uvinul®T150)
phenol, 2-(2H-benzotriazol-2-yl)-4-methyl-6-(2-methyl-3(1,3,3,3-tetramethyl-1-(trimethylsilyl)oxy)disiloxyanyl) propyl) (Mexoryl®XL)
4,4′-[(6-[4-(1,1-dimethyl)aminocarbonyl)phenylamino]-1,3,5-triazine-2,4-diyl)diimino]-bis-(benzoic acid-2-ethylhexyl ester) (Uvasorb HEB)
3-(4′-methyl benzylidene)-d,l-camphor (Neo Heliopan®MBC)
3-benzylidene camphor
salicylic acid-2-ethylhexyl ester (Neo Heliopan®OS)
4-dimethylaminobenzoic acid-2-ethylhexyl ester (Padimate O)
hydroxy-4-methoxybenzophenone-5-sulfonic acid and Na salt
2,2′-methylene bis-(6-(2H-benzotriazol-2-yl)-4-1,1,3,3-tetramethylbutyl)phenol) (Tinosorb®M)
phenylene bis-benzimidazyl tetrasulfonic acid disodium salt (Neo Heliopan®AP)
2,4-bis-[{(4-(2-ethyl hexyloxy)-2-hydroxy}phenyl]-6-(4-methoxyphenyl)-1,3,5-triazine (Tinosorb®S)
benzylidene malonate polysiloxane (Parsol®SLX)
menthyl anthranilate (Neo Heliopan®MA)
2-(4-diethylamino-2-hydroxybenzoyl)benzoic acid hexyl ester (Uvinul® A Plus)
indanylidene compounds in accordance with DE 100 55 940 (=WO 02/38537)

Advantageous inorganic light protection pigments are finely dispersed metal oxides and metal salts, for example titanium dioxides, zinc oxide (ZnO), iron oxides (e.g. Fe₂O₃), aluminium oxide (Al₂O₃); cerium oxides (e.g. Ce₂O₃), manganese oxides (e.g. MnO), zirconium oxide (ZrO₂), silicon oxide (SiO₂), mixed oxides of the corresponding metals and mixtures of such oxides, barium sulfate and zinc stearate. Pigments based on TiO₂ or zinc oxide are particularly preferred. In preferred embodiments the particles have an average diameter of less than 100 nm, preferably between 5 and 50 nm and particularly preferably between 15 and 30 nm. They can display a spherical form, but such particles having an ellipsoid form or other form deviating from the spherical shape can also be used. The pigments can also be surface treated, i.e. hydrophilised or hydrophobed. Typical examples are coated titanium dioxides, such as e.g. titanium dioxide T 805 (Degussa) or Eusolex® T2000 (Merck) or coated zinc oxide, such as e.g. zinc oxide NDM. Suitable hydrophobic coating agents are above all silicones and especially trialkoxyoctyl silanes or simethicones. So-called micro-pigments or nano-pigments are preferably used in sunscreens. Zinc micro- or nano-pigments are preferably used.

The total amount of inorganic pigments, particularly hydrophobic inorganic micro-pigments, in the finished cosmetic or dermatological formulations is advantageously in the range from 0.1 to 30 wt. %, preferably 0.1 to 10.0, in particular 0.5 to 6.0 wt. %, relative to the total weight of the formulations.

The formulations according to the invention can also contain antioxidants, wherein all antioxidants that are suitable for or commonly used for cosmetic and/or dermatological applications can be used. The antioxidants are advantageously selected from the group consisting of amino acids (e.g. glycine, histidine, tyrosine, tryptophane) and derivatives thereof, imidazoles (e.g. urocanic acid) and derivatives thereof, peptides such as D, L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propyl thiouracil and other thiols (e.g. thioredoxin, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and the salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximine, homocysteine sulfoximine, buthionine sulfone, penta-, hexa-, hepta-thionine sulfoximine) in very small compatible doses, also (metal) chelators, e.g. α-hydroxy fatty acids, palmitic acid, phytic acid, lactoferrin, α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate, ascorbyl glycosides such as e.g. 6-O-acyl-2-O-α-D-glucopyranosyl-L-ascorbic acid, 6-O-acyl-2-O-β-D-glucopyranosyl-L-ascorbic acid, 2-O-α-D-glucopyranosyl-L-ascorbic acid or 2-O-β-D-glucopyranosyl-L-ascorbic acid), tocopherols and derivatives thereof (e.g. vitamin E acetate), vitamin A and derivatives thereof (vitamin A palmitate) as well as coniferyl benzoate of benzoic resin, rutic acid and derivatives thereof, α-glucosyl rutin, quercetin and derivatives thereof, rosemarinic acid, carnosol, carnosolic acid, resveratrol, caffeic acid and derivatives thereof, sinapic acid and derivatives thereof, ferulic acid and derivatives thereof, furfurylidene glucitol, curcuminoids, butyl hydroxytoluene, butyl hydroxyanisole, nordihydroguaiacic resin acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO₄) selenium and derivatives thereof (e.g. selenium methionine), stilbenes and derivatives thereof (e.g. stilbene oxide, trans-stilbene oxide) along with derivatives (salts, esters, ethers, sugars, nucleotides, nucleosides, peptides and lipids) of these cited active ingredients or extracts or fractions of plants having an antioxidant effect, such as e.g. green tea, rooibos, honeybush, grape, rosemary, sage, melissa, thyme, lavender, olive, oats, cocoa, ginkgo, ginseng, liquorice, honeysuckle, sophora, pueraria, pinus, citrus, Phyllanthus emblica or St. John's wort.

The amount of antioxidants (one or more compounds) in the formulations according to the invention is preferably 0.01 to 20 wt. %, particularly preferably 0.05 to 10 wt. %, in particular 0.2 to 5 wt. %, relative to the total weight of the preparation.

If vitamin E and/or derivatives thereof are used as the antioxidant(s), it is advantageous to choose their concentrations from the range from 0.001 to 10 wt. %, relative to the total weight of the formulation.

If vitamin A or vitamin A derivatives or carotenes or derivatives thereof are used as the antioxidant(s), it is advantageous to choose their concentrations from the range from 0.001 to 10 wt. %, relative to the total weight of the formulation.

The (cosmetic) formulations according to the invention can also contain active ingredients and combinations of active ingredients to combat skin ageing and wrinkles. All active ingredients that are suitable for or commonly used for cosmetic and/or dermatological applications to combat skin ageing and wrinkles can be used here according to the invention. Advantageous active ingredients in this respect to combat skin ageing and wrinkles are soya protein or protein hydrolysates, soya isoflavones, hydrolysed rice protein, hydrolysed hazelnut protein, oligopeptides from hydrolysed Hibiscus esculentus extract, wheat protein, β-glucanes e.g. from oats and derivatives thereof, glycoproteins, ursolic acid and salts thereof, betulin, betulinic acid and salts thereof, retinol, retinol palmitate, propyl gallate, precocene, 6-hydroxy-7-methoxy-2,2-dimethyl-1(2H)-benzopyran, 3,4-dihydro-6-hydroxy-7-methoxy-2,2-dimethyl-1(2H)-benzopyran, creatine or other synthetic or natural active ingredients to combat skin ageing and wrinkles, wherein the latter can also be used in the form of an extract from plants, such as e.g. green tea, Rubus fruticosus, Sanguisorba officinalis, Centella asiatica, Ribes nigrum, Passiflora incarnata, Phyllanthus emblica, okra, algae, evening primrose, rosemary, sage, echinacea, birch, apple or soya.

Particularly preferred for use as additional active ingredients to combat skin ageing is β-glucane, wherein 1,3-1,4-coupled β-glucane from oats, Rubus fruticosus extract or wheat protein is especially preferred.

The use of anti-inflammatory active ingredients and/or active ingredients to relieve reddening and/or itching is also advantageous in the formulations according to the invention. All anti-inflammatory active ingredients or active ingredients to relieve reddening and/or itching which are suitable for or commonly used for cosmetic and/or dermatological applications can be used here. Steroidal anti-inflammatory substances of the corticosteroid type, such as e.g. hydrocortisone, hydrocortisone derivatives such as hydrocortisone-17-butyrate, dexamethasone, dexamethasone phosphate, methyl prednisolone or cortisone, are advantageously used as anti-inflammatory active ingredients or active ingredients to relieve reddening and/or itching, the list of which can be extended by the addition of other steroidal anti-inflammatories. Non-steroidal anti-inflammatories can also be used. Examples which can be cited here are oxicams such as piroxicam or tenoxicam; salicylates such as aspirin, disalcid, solprin or fendosal; acetic acid derivatives such as diclofenac, fenclofenac, indomethacin, sulindac, tolmetin or clindanac; fenamates such as mefenamic, meclofenamic, flufenamic or niflumic; propionic acid derivatives such as ibuprofen, naproxen, benoxaprofen or pyrazoles such as phenylbutazone, oxyphenylbutazone, febrazone or azapropazone. Alternatively, natural anti-inflammatory substances or substances to relieve reddening and/or itching can be used. Plant extracts, special highly active plant extract fractions and highly pure active substances isolated from plant extracts can be used. Particularly preferred are extracts, fractions and active substances from camomile, aloe vera, commiphora species, rubia species, ginger, willow, willowherb, oats, calendula, arnica, St. John's wort, honeysuckle, rosemary, Passiflora incarnata, witch hazel, avena, dianthus or echinacea, as well as pure substances such as inter alia bisabolol, apigenin, apigenin-7-glucoside, boswellic acid, phytosterols, glycyrrhizinic acid, glabridin, licochalcone A and anthranilic acid amides such as in particular avenanthramides or dianthramides. The formulations according to the invention can also contain mixtures of two or more anti-inflammatory active ingredients.

The amount of anti-irritants (one or more compounds) in the preparations is preferably 0.0001 to 20 wt. %, particularly preferably 0.0001 to 10 wt. %, in particular 0.001 to 5 wt. %, relative to the total weight of the preparation.

Formulations according to the invention can advantageously also contain moisture regulators. The following substances, for example, can be used as moisture regulators (moisturisers): sodium lactate, urea, urea derivatives, alcohols, glycerol, diols such as propylene glycol, 1,2-pentanediol, 1,2-hexanediol and 1,2-octanediol, collagen, elastin or hyaluric acid, diacyl adipates, petroleum jelly, urocanic acid, lecithin, panthenol, phytanetriol, lycopene, (pseudo)ceramides, glycosphingolipids, cholesterol, phytosterols, chitosan, chondroitin sulfate, lanolin, lanolin esters, amino acids, alpha-hydroxy acids (e.g. citric acid, lactic acid, malic acid) and derivatives thereof, mono-, di- and oligosaccharides such as e.g. glucose, galactose, fructose, mannose, fruit sugars and lactose, poly sugars such as β-glucanes, in particular 1,3-1,4-β-glucane from oats, alpha-hydroxy fatty acids, triterpene acids such as betulinic acid or ursolic acid, algal extracts.

The glycosyl flavanones for use according to the invention can advantageously be used together with osmolytes. Examples of osmolytes which can be cited are: substances from the group of sugar alcohols (myo-inositol, mannitol, sorbitol), quaternary amines such as taurine, choline, betaine, betaine glycine, ectoine, diglycerol phosphate, phosphorylcholine, glycerophosphorylcholines, amino acids such as glutamine, glycine, alanine, glutamate, aspartate or proline, phosphatidylcholine, phosphatidylinositol, inorganic phosphates, and polymers of the cited compounds such as proteins, peptides, polyamino acids and polyols. All osmolytes also have a skin-moistening action.

Formulations according to the invention containing glycosyl flavanones can also contain anionic, cationic, non-ionic and/or amphoteric surfactants, especially if crystalline or microcrystalline solids, for example inorganic micropigments, are to be incorporated into the preparations.

Anionic surfactants generally display carboxylate, sulfate or sulfonate groups as functional groups. In aqueous solution they form negatively charged organic ions in the acid or neutral environment. Cationic surfactants are almost exclusively characterised by the presence of a quaternary ammonium group. In aqueous solution they form positively charged organic ions in the acid or neutral environment. Amphoteric surfactants contain both anionic and cationic groups and therefore behave in aqueous solution in the same way as anionic or cationic surfactants, depending on the pH. They have a positive charge in a strongly acid environment and a negative charge in an alkaline environment. In the neutral pH range, by contrast, they are zwitterionic. Polyether chains are typical of non-ionic surfactants. Non-ionic surfactants do not form ions in the aqueous medium.

### A. Anionic Surfactants

Anionic surfactants which can advantageously be used are acyl amino acids (and salts thereof), such as
acyl glutamates, for example sodium acyl glutamate, di-TEA-palmitoyl aspartate and sodium caprylic/capric glutamate,
acyl peptides, for example palmitoyl-hydrolysed milk protein, sodium cocoyl-hydrolysed soya protein and sodium/potassium cocoyl-hydroylsed collagen,
sarcosinates, for example myristoyl sarcosin, TEA-lauroyl sarcosinate, sodium lauroyl sarcosinate and sodium cocoyl sarcosinate,
taurates, for example sodium lauroyl taurate and sodium methyl cocoyl taurate,
acyl lactylates, lauroyl lactylate, caproyl lactylate
alaninates
   carboxylic acid and derivatives, such as
   for example lauric acid, aluminium stearate, magnesium alkanolate and zinc undecylenate,
ester carboxylic acids, for example calcium stearoyl lactylate, laureth-6 citrate and sodium PEG-4 lauramide carboxylate,
ether carboxylic acids, for example sodium laureth-13 carboxylate and sodium PEG-6 cocamide carboxylate,
   phosphoric acid esters and salts, such as e.g. DEA-oleth-10-phosphate and dilaureth-4 phosphate,
   sulfonic acids and salts, such as
acyl isothionates, e.g. sodium/ammonium cocoyl isethionate,
alkyl aryl sulfonates,
alkyl sulfonates, for example sodium cocomonoglyceride sulfate, sodium C₁₂₋₁₄ olefin sulfonate, sodium lauryl sulfoacetate and magnesium PEG-3 cocamide sulfate,
sulfosuccinates, for example dioctyl sodium sulfosuccinate, disodium laureth sulfosuccinate, disodium lauryl sulfosuccinate and disodium undecylenamido MEA sulfosuccinate
   and
   sulfuric acid esters, such as
alkyl ether sulfate, for example sodium, ammonium, magnesium, MIPA, TIPA laureth sulfate, sodium myreth sulfate and sodium C₁₂₋₁₃ pareth sulfate,
alkyl sulfates, for example sodium, ammonium and TEA lauryl sulfate.

### B. Cationic Surfactants

Cationic surfactants which can advantageously be used are
alkyl amines,
alkyl imidazoles,
ethoxylated amines and
quaternary surfactants.
   RNH₂CH₂CH₂COO⁻ (where pH=7)
   RNHCH₂CH₂COO—B⁺ (where pH=12) B⁺=any cation, e.g. Na⁺
esterquats

Quaternary surfactants contain at least one N atom, which is covalently bonded to 4 alkyl or aryl groups. This leads to a positive charge, regardless of the pH. Alkyl betaine, alkyl amidopropyl betaine and alkyl amidopropyl hydroxysulfaine are advantageous. The cationic surfactants used can also preferably be chosen from the group of quaternary ammonium compounds, in particular benzyl trialkyl ammonium chlorides or bromides, such as benzyl dimethylstearyl ammonium chloride for example, also alkyl trialkyl ammonium salts, for example cetyl trimethyl ammonium chloride or bromide, alkyl dimethyl hydroxyethyl ammonium chlorides or bromides, dialkyl dimethyl ammonium chlorides or bromides, alkyl amide ethyl trimethyl ammonium ether sulfates, alkyl pyridinium salts, for example lauryl or cetyl pyrimidinium chloride, imidazoline derivatives and compounds having a cationic character such as amine oxides, for example alkyl dimethyl amine oxides or alkyl aminoethyl dimethyl amine oxides. Cetyl trimethyl ammonium salts are particularly advantageously used.

### C. Amphoteric Surfactants

Amphoteric surfactants which can advantageously be used are
acyl/dialkyl ethylene diamine, for example sodium acyl amphoacetate, disodium acyl amphodipropionate, disodium alkyl amphodiacetate, sodium acyl amphohydroxypropyl sulfonate, disodium acyl amphodiacetate and sodium acyl amphopropionate,
N-alkyl amino acids, for example aminopropyl alkyl glutamide, alkyl aminopropionic acid, sodium alkyl imidodipropionate and lauroamphocarboxyglycinate.

### D. Non-Ionic Surfactants

Non-ionic surfactants which can advantageously be used are
alcohols,
alkanolamides, such as cocamides MEA/DEA/MIPA,
amine oxides, such as cocamidopropylamine oxide,
esters produced by esterification of carboxylic acids with ethylene oxide, glycerol, sorbitan or other alcohols,
ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides such as lauryl glucoside, decyl glycoside and cocoglycoside.
sucrose esters, ethers
polyglycerol esters, diglycerol esters, monoglycerol esters
methyl glucose esters, esters of hydroxy acids

The use of a combination of anionic and/or amphoteric surfactants with one or more non-ionic surfactants is also advantageous.

The surface-active substance can be present in formulations according to the invention in a concentration of between 1 and 98 wt. %, relative to the total weight of the preparations.

A lipid phase in formulations according to the invention can advantageously be chosen from the following groups of substances:
mineral oils (advantageously paraffin oil), mineral waxes
fatty oils, fats, waxes and other natural and synthetic fat bodies, preferably esters of fatty acids with low C-number alcohols, for example with isopropanol, propylene glycol or glycerol, or esters of fatty alcohols with low C-number alkanoic acids or with fatty acids;
alkyl benzoates;
silicone oils such as dimethyl polysiloxanes, diethyl polysiloxanes, diphenyl polysiloxanes and mixed forms thereof
hydrocarbons (advantageously squalane or squalene)
synthetic or semisynthetic triglyceride oils (e.g. triglycerides of capric or caprylic acid)
natural oils (one or more conditioning animal and/or vegetable fats and oils such as olive oil, sunflower oil, refined soya oil, palm oil, sesame oil, rapeseed oil, almond oil, borage oil, evening primrose oil, coconut butter, shea butter, jojoba oil, oat oil, sperm oil, beef fat, neatsfoot oil and pig fat)
   and optionally other conditioning constituents such as e.g. fatty alcohols having 8-30 C atoms. The fatty alcohols here can be saturated or unsaturated and linear or branched. Examples that can be used include decanol, decenol, octanol, octenol, dodecanol, dodecenol, octadienol, decadienol, dodecadienol, oleyl alcohol, ricinol alcohol, erucic alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well as Guerbet alcohols thereof, wherein the list could be extended almost at will with other alcohols having a related chemical structure. The fatty alcohols preferably come from natural fatty acids, being conventionally produced from the corresponding esters of the fatty acids by reduction. Also usable are fatty alcohol fractions produced by reduction from naturally occurring fats and fatty oils, such as e.g. beef fat, groundnut oil, colza oil, cottonseed oil, soya bean oil, sunflower oil, palm kernel oil, linseed oil, maize oil, castor oil, rapeseed oil, sesame oil, cocoa butter and coconut butter. Synthetic ester oils can also be included. Preferred examples are esters of saturated and/or unsaturated, linear and/or branched alkane carboxylic acids having 3 to 30 C atoms with saturated and/or unsaturated, linear and/or branched alcohols having 3 to 30 C atoms and esters of aromatic carboxylic acids with saturated and/or unsaturated, linear and/or branched alcohols having 3 to 30 atoms, selected in particular from the group comprising isopropyl myristate, isopropyl stearate, isopropyl palmitate, isopropyl oleate, n-butyl stearate, n-hexyl laurate, n-decyl laurate, isooctyl stearate, isononyl stearate, isononyl isononanoate, 2-ethylhexyl palmitate, 2-ethylhexyl laurate, 2-ethylhexyl ethylhexanoate, cetearyl-2-ethylhexanoate, 3,5,5-trimethylhexyl-3,5,5-trimethyl hexanoate, 2-ethylhexyl isononanoate, 2-ethylhexyl-3,5,5-trimethyl hexanoate, 2-ethylhexyl-2-ethylhexanoate, 2-hexyl decyl stearate, 2-octyl decyl palmitate, oleyl oleate, oleyl erucate, erucyl oleate, erucyl erucate and synthetic or natural mixtures of such esters), fats, waxes and other natural and synthetic fat bodies, preferably esters of fatty alcohols with low C-number alcohols (e.g. with isopropanol, propylene glycol or glycerol) or esters of fatty alcohols with low C-number alkanoic acids or with fatty acids, alkyl benzoates (e.g. mixtures of n-dodecyl, n-tridecyl, n-tetradecyl and n-pentadecyl benzoate) and cyclic or linear silicone oils (such as e.g. dimethyl polysiloxanes, diethyl polysiloxanes, diphenyl polysiloxanes and mixed forms thereof).

Other conditioning substances which combine well with glycosyl flavanones include
waxes such as e.g. candelilla wax or carnauba wax
ceramides, wherein ceramides are understood to be N-acyl sphingosines (fatty acid amides of sphingosine) or synthetic analogues of such lipids (so-called pseudoceramides), which markedly improve the water-retaining capacity of the stratum corneum.
phospholipids, for example soya lecithin, egg lecithin and kephalins
vaseline, paraffin and silicone oils; the latter include inter alia dialkyl and alkylaryl siloxanes such as dimethyl polysiloxane and methylphenyl polysiloxane, as well as alkoxylated and quaternised derivatives thereof.

An aqueous phase in formulations according to the invention can advantageously include: alcohols, diols or polyols having a low C number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, also alcohols having a low C number, e.g. ethanol, isopropanol, 1,2-propanediol, glycerol and in particular one or more thickeners, which can advantageously be chosen from the group comprising silicon dioxide, aluminium silicates, polysaccharides or derivatives thereof, e.g. hyaluronic acid, xanthan gum, hydroxypropyl methyl cellulose, particularly advantageously from the group of polyacrylates, preferably a polyacrylate from the group of so-called carbopols, for example type 980, 981, 1382, 2984, 5984 carbopols, either individually or in combination.

Formulations according to the invention in the form of an emulsion advantageously include one or more emulsifiers. O/W emulsifiers, for example, can advantageously be chosen from the group of polyethoxylated or polypropoxylated or polyethoxylated and polypropoxylated products, e.g.:
fatty alcohol ethoxylates
ethoxylated wool wax alcohols,
polyethylene glycol ethers having the general formula R—O—(—CH₂—CH₂—O—)ₙ—R′,
fatty acid ethoxylates having the general formula R—COO—(—CH₂—CH₂—O—)ₙ—H,
etherified fatty acid ethoxylates having the general formula

R—COO—(—CH₂—CH₂—O—)ₙ—R′,
esterified fatty acid ethoxylates having the general formula

R—COO—(—CH₂—CH₂—O—)ₙ—C(O)—R′,
polyethylene glycol glycerol fatty acid esters
ethoxylated sorbitan esters,
cholesterol ethoxylates
ethoxylated triglycerides
alkyl ether carboxylic acids having the general formula

R—COO—(—CH₂—CH₂—O—)ₙ—OOH, where n represents a number from 5 to 30,
polyoxyethylene sorbitol fatty acid esters,
alkyl ether sulfates having the general formula R—O—(—CH₂—CH₂—O—)ₙ—SO₃—H
fatty alcohol propoxylates having the general formula R—O—(—CH₂—CH(CH₃)—O—)ₙ—H
polypropylene glycol ethers having the general formula

R—O—(—CH₂—CH(CH₃)—O—)ₙ—R′
propoxylated wool wax alcohols,
etherified fatty acid propoxylates R—COO—(—CH₂—CH(CH₃)—O—)ₙ—R′
esterified fatty acid propoxylates having the general formula

R—COO—(—CH₂—CH(CH₃)—O—)ₙ—C(O)—R′
fatty acid propoxylates having the general formula

R—COO—(—CH₂—CH(CH₃)—O—)ₙ—H,
polypropylene glycol glycerol fatty acid esters
propoxylated sorbitan esters,
cholesterol propoxylates
propoxylated triglycerides,
alkyl ether carboxylic acids having the general formula

R—O—(—CH₂—CH(CH₃)—O—)ₙ—CH₂—COOH,
alkyl ether sulfates or the acids underlying these sulfates having the general formula R—O—(—CH₂—CH(CH₃)—O—)ₙ—SO₃—H,
fatty alcohol ethoxylates/propoxylates having the general formula R—O—Xₙ—Yₘ—H
polypropylene glycol ethers having the general formula R—O—Xₙ—Yₘ—R′
etherified fatty acid propoxylates having the general formula R—COO—Xₙ—Yₘ—R′
fatty acid ethoxylates/propoxylates having the general formula R—COO—Xₙ—Yₘ—H.

Particularly advantageously according to the invention the polyethoxylated or polypropoxylated or polyethoxylated and polypropoxylated O/W emulsifiers used are chosen from the group of substances having HLB values of 11 to 18, most particularly advantageously having HLB values of 14.5 to 15.5, if the O/W emulsifiers display saturated R and R′ radicals. If the O/W emulsifiers display unsaturated R and/or R′ radicals, or if isoalkyl derivatives are present, the preferred HLB value of such emulsifiers can also be lower or higher.

It is advantageous to choose the fatty alcohol ethoxylates from the group of ethoxylated stearyl alcohols, cetyl alcohols, cetyl stearyl alcohols (cetearyl alcohols). Particularly preferred are:

Polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is also advantageous to choose the fatty acid ethoxylates from the following group:

Polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

Sodium laureth-11 carboxylate can advantageously be used as the ethoxylated alkyl ether carboxylic acid or its salt. Sodium laureth 1-4 sulfate can advantageously be used as the alkyl ether sulfate. Polyethylene glycol (30) cholesteryl ether can advantageously be used as the ethoxylated cholesterol derivative. Polyethylene glycol (25) soya sterol has also proved itself.

Polyethylene glycol (60) evening primrose glycerides can advantageously be used as ethoxylated triglycerides.

It is also advantageous to choose the polyethylene glycol glycerol fatty acid esters from the group comprising polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise advantageous to choose the sorbitan esters from the group comprising polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be used as advantageous W/O emulsifiers: fatty alcohols having 8 to 30 carbon atoms, monoglycerol esters of saturated and/or unsaturated, branched and/or unbranched alkane carboxylic acids having a chain length of 8 to 24, in particular 12 to 18 C atoms, diglycerol esters of saturated and/or unsaturated, branched and/or unbranched alkane carboxylic acids having a chain length of 8 to 24, in particular 12 to 18 C atoms, monoglycerol ethers of saturated and/or unsaturated, branched and/or unbranched alcohols having a chain length of 8 to 24, in particular 12 to 18 C atoms, diglycerol ethers of saturated and/or unsaturated, branched and/or unbranched alcohols having a chain length of 8 to 24, in particular 12 to 18 C atoms, propylene glycol esters of saturated and/or unsaturated, branched and/or unbranched alkane carboxylic acids having a chain length of 8 to 24, in particular 12 to 18 C atoms and sorbitan esters of saturated and/or unsaturated, branched and/or unbranched alkane carboxylic acids having a chain length of 8 to 24, in particular 12 to 18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate.

Formulations according to the invention (e.g. topical cosmetic formulations) advantageously contain cooling agents. Examples of cooling agents which can be cited are: l-menthol, d-menthol, racemic menthol, menthone glycerine acetal, menthyl lactate, substituted menthyl-3-carboxylic acid amides (e.g. menthyl-3-carboxylic acid-N-ethylamide), 2-isopropyl-N-2,3-trimethyl butanamide, substituted cyclohexane carboxylic acid amides, 3-menthoxypropane-1,2-diol, 2-hydroxyethyl menthyl carbonate, 2-hydroxypropyl menthyl carbonate, N-acetyl glycine menthyl ester, isopulegol, menthyl hydroxycarboxylic acid esters (e.g. menthyl-3-hydroxybutyrate), monomenthyl succinate, 2-mercaptocyclodecanone, menthyl-2-pyrrolidin-5-one carboxylate, 2,3-dihydroxy-p-menthane, 3,3,5-trimethyl cyclohexanone glycerine ketal, 3-menthyl-3,6-di- and trioxaalkanoates, 3-menthyl methoxyacetate, icilin.

The formulations according to the invention (e.g. topical cosmetic formulations) also advantageously contain antimicrobial active ingredients. Worth mentioning in addition to standard preservatives as further active ingredients are in particular, in addition to the large group of standard antibiotics, the products relevant for cosmetics, such as triclosan, climbazole, zinc pyrithione, ichthyol, octopirox (1-hydroxy-4-methyl-6-(2,4,4-trimethylpentyl)-2(1H)-pyridones, 2-aminoethanol), chitosan, farnesol, octoxyglycerine, glycerol monolaurate, aryl alkyl alcohols such as e.g. phenylethyl alcohol, 3-phenyl-1-propanol, veticol or muguet alcohol and aliphatic diols such as e.g. 1,2-decanediol or combinations of the cited substances, which are used inter alia against underarm odour, foot odour or dandruff formation.

Also suitable for use are:

Aryl- or aryloxy-substituted, unbranched or monoalkyl- and polyalkyl-branched saturated or unsaturated
fatty alcohols, aldehydes and acids, acid esters
alkane diols, dialdehydes and dicarboxylic acids and esters
   having chain lengths of C₂ to C₄₀, from synthetic or natural sources (e.g. from coconut butter, palm kernel oil, wool wax, lanolin);

Monohydroxy and oligohydroxy fatty acids having chain lengths of C₂ to C₂₄ (e.g. lactic acid, 2-hydroxypalmitic acid), oligomers and/or polymers thereof and plant and animal raw materials containing these;

Ethoxylated, propoxylated or mixed ethoxylated/propoxylated cosmetic fatty alcohols, fatty acids and fatty acid esters having chain lengths of C₂ to C₄₀ having 1 to 150 E/O and/or P/O units.

So-called “natural” antibacterial active ingredients can also be used, most of which are essential oils. Typical oils having an antibacterial action are, for example, oils of aniseed, lemon, orange, rosemary, wintergreen, clove, thyme, lavender, hops, citronella, wheat, lemongrass, cedarwood, cinnamon, geranium, sandalwood, violet, eucalyptus, peppermint, gum benzoin, basil, fennel and Ocmea origanum, Hydastis carradensis, Berberidaceae daceae, Ratanhiae or Curcuma longa.

Important substances having an antimicrobial action which can be found in essential oils are for example anethol, catechol, camphene, carvacrol, eugenol, eucalyptol, ferulic acid, farnesol, hinokitiol, tropolone, limonene, menthol, methyl salicylate, thymol, terpineol, verbenone, berberine, curcumin, caryophyllene oxide, nerolodol, geraniol.

Mixtures of the cited active systems or active ingredients and active ingredient combinations containing these active ingredients can also be used.

The amount of active ingredients in the preparations is preferably 0.01 to 20 wt. %, relative to the total weight of the preparations, particularly preferably 0.05 to 10 wt. %.

The glycosyl flavanones having formula (I) for use according to the invention can moreover also be used in combination with sweat-inhibiting active ingredients (antiperspirants) and odour absorbers. Aluminium salts above all such as aluminium chloride, aluminium chlorohydrate, nitrate, sulfate, acetate, etc., but also aluminium hydroxychlorides, can be used as sweat-inhibiting active ingredients. The use of zinc, magnesium and zirconium compounds can also be advantageous, however. The following can also be used: a) protein-precipitating substances such as inter alia formaldehyde, glutaraldehyde, natural and synthetic tannins and trichloroacetic acid, which bring about a surface closure of the sweat glands, b) local anaesthetics (including dilute solutions of e.g. lidocaine, prilocalne or mixtures of such substances), which switch off the sympathic supply to the sweat glands by blocking the peripheral nerves, c) type X, A or Y zeolites which in addition to reducing sweat secretion also act as adsorbing agents for unpleasant odours, and d) botulinus toxin (toxin of the bacterium Chlostridium botulinum), and other substances which bring about a blocking of the release of the transmitter substance acetyl choline which is relevant for sweat secretion.

Odour absorbers are for example the phyllosilicates described in DE 40 09 347, in particular montmorillonite, kaolinite, nontronite, saponite, hectorite, bentonite, smectite, and also zinc salts of ricinoleic acid for example. They also include deodorants, bactericidal or bacteriostatic deodorising substances, such as e.g. hexachlorophene, 2,4,4′-trichloro-2′-hydroxydiphenyl ether (Irgasan), 1,6-di-(4-chlorophenylbiguanido)hexane (chlorhexidine), 3,4,4′-trichlorocarbanilide, and the active agents described in DE 37 40 186, DE 39 38 140, DE 42 04 321, DE 42 29 707, DE 42 29 737, DE 42 37 081, DE 43 09 372, DE 43 24 219 and containing cation-active substances, such as e.g. quaternary ammonium salts and odour absorbers such as e.g. Grillocin® (combination of zinc ricinoleate and various additives) or triethyl citrate, optionally in combination with ion-exchange resins.

The amount of deodorising and/or antiperspirant active ingredients in the formulations is preferably 0.01 to 20 wt. %, relative to the total weight of the preparations, particularly preferably 0.05 to 10 wt. %.

The glycosyl flavanones for use according to the invention can also in many cases advantageously be used in combination with preservatives. Preservatives chosen here are preferably those such as benzoic acid, esters and salts thereof, propionic acid and salts thereof, salicylic acid and salts thereof, 2,4-hexadienoic acid (sorbic acid) and salts thereof, formaldehyde and paraformaldehyde, 2-hydroxybiphenyl ether and salts thereof, 2-zinc sulfidopyridine-N-oxide, inorganic sulfites and bisulfites, sodium iodate, chlorobutanol, 4-ethyl mercury(II)-5-amino-1,3-bis(2-hydroxybenzoic acid, salts and esters thereof, dehydracetic acid, formic acid, 1,6-bis(4-amidino-2-bromophenoxy)-n-hexane and salts thereof, the sodium salt of ethyl mercury(II)-thiosalicylic acid, phenyl mercury and salts thereof, 10-undecenoic acid and salts thereof, 5-amino-1,3-bis(2-ethylhexyl)-5-methyl-hexahydropyrimidine, 5-bromo-5-nitro-1,3-dioxan, 2-bromo-2-nitro-1,3-propanediol, 2,4-dichlorobenzyl alcohol, N-(4-chlorophenyl)-N′-(3,4-dichlorophenyl)urea, 4-chloro-m-cresol, 2,4,4′-trichloro-2′-hydroxydiphenyl ether, 4-chloro-3,5-dimethyl phenol, 1,1′-methylene-bis(3-(1-hydroxymethyl-2,4-dioximidazolidin-5-yl)urea), poly(hexamethylene diguanide)hydrochloride, 2-phenoxyethanol, hexamethylene tetramine, 1-(3-chloroallyl)-3,5,7-triaza-1-azoniaadamantane chloride, 1-(4-chlorophenoxy)-1-(1H-imidazol-1-yl)-3,3-dimethyl-2-butanone, 1,3-bis-(hydroxymethyl)-5,5-dimethyl-2,4-imidazolidinedione, benzyl alcohol, octopirox, 1,2-dibromo-2,4-dicyanobutane, 2,2′-methylene-bis(6-bromo-4-chlorophenol), bromochlorophene, mixture of 5-chloro-2-methyl-3(2H)-isothiazolinone and 2-methyl-3(2H)-isothiazolinone with magnesium chloride and magnesium nitrate, 2-benzyl-4-chlorophenol, 2-chloroacetamide, chlorhexidine, chlorhexidine acetate, chlorhexidine gluconate, chlorhexidine hydrochloride, 1-phenoxypropan-2-ol, N-alkyl-(C₁₂-C₂₂)-trimethyl-ammonium bromide and chloride, 4,4-dimethyl-1,3-oxazolidine, N-hydroxymethyl-N-(1,3-di(hydroxymethyl)-2,5-dioxoimidazolidin-4-yl)-N′-hydroxymethyl urea, 1,6-bis(4-amidinophenoxy)-n-hexane and salts thereof, glutaraldehyde, 5-ethyl-1-aza-3,7-dioxabicyclo(3.3.0)octane, 3-(4-chlorophenoxy)-1,2-propanediol, hyamine, alkyl-(C₈-C₁₈)-dimethylbenzyl ammonium chloride, alkyl-(C₈-C₁₈)-dimethylbenzyl ammonium bromide, alkyl-(C₈-C₁₈)-dimethylbenzyl ammonium saccharinate, benzyl hemiformal, 3-iodine-2-propinyl butyl carbamate, sodium hydroxymethylamino acetate or sodium hydroxymethylamino acetate.

Formulations according to the invention, in particular dermatological formulations, can also advantageously contain dyes and/or coloured pigments, particularly if they are intended for use in the area of decorative cosmetics. The dyes and coloured pigments can be selected from the corresponding positive list in the German cosmetics ordinance or the EU list of cosmetic colorants. In most cases they are identical to the dyes approved for foodstuffs. Advantageous coloured pigments are for example titanium dioxide, mica, iron oxides (e.g. Fe₂O₃ Fe₃O₄, FeO(OH)) and/or tin oxide. Advantageous dyes are for example carmine, Berlin blue, chromium oxide green, ultramarine blue and/or manganese violet.

Mixtures of the cited active systems can also be used.

For use, topical formulations according to the invention are applied to the skin and/or hair in an adequate amount in the conventional way for cosmetics, for skin and hair browning.

Other preferred embodiments of the invention can be seen from the following examples and the appended claims:

### EXAMPLE 1

### Skin-Browning Oil-in-Water Emulsion

| | Raw material name | | |
|---|---|---|---|
| Part | (manufacturer) | INCI name | Content in wt. % |
| A | Dragosan W/O P (Symrise) | Sorbitan isostearate, hydrogenated | 6.00 |
| | | castor oil, ceresin, beeswax (Cera alba) | |
| | PCL-Liquid (Symrise) | Cetearyl ethylhexanoate, isopropyl | 12.00 |
| | | myristate | |
| | Sunflower oil (H. Erhard | Helianthus annuus (sunflower) seed oil | 5.00 |
| | Wagner) | | |
| | Sweet almond oil (H. Erhard | Prunus dulcis | 5.00 |
| | Wagner) | | |
| | Dragosan W/O Liquid | Polyglyceryl-3-polyricinoleate, sorbitan | 1.00 |
| | (Symrise) | isostearate | |
| | Alugel 34 TH (Baerlocher) | Aluminium stearate | 1.00 |
| | Oxynex 2004 (Merck) | BHT | 0.10 |
| B | Demineralised water | Water (aqua) | 56.20 |
| | Glycerine, 99.5% | Glycerine | 2.00 |
| | Karion F (Merck) | Sorbitol | 2.00 |
| | Aloe Vera Gel Concentrate | Water (aqua), Aloe barbadensis leaf juice | 3.00 |
| | 10/1 (Symrise) | | |
| | Extrapone Hamamelis | Propylene glycol, Hamamelis virginiana | 1.00 |
| | distillate, colourless | (witch hazel) water, water (aqua), | |
| | (Symrise) | Hamamelis virginiana (witch hazel) | |
| | | extract | |
| | Magnesium sulfate | Magnesium sulfate | 0.70 |
| | heptahydrate (Merck) | | |
| | Dragocid Liquid (Symrise) | Phenoxyethanol, methylparaben, | 0.80 |
| | | ethylparaben, butylparaben, | |
| | | propylparaben, isobutylparaben | |
| C | Vitamin E acetate (DSM | Tocopheryl acetate | 3.00 |
| | Nutritional Products) | | |
| | Vitamin A palmitate in oil (1 | Retinyl palmitate | 0.20 |
| | million le/G) (DSM | | |
| | Nutritional Products) | | |
| | Naringin | 4′,5,7-Trihydroxyflavone-7-O- | 0.50 |
| | | neohesperidoside | |
| | -(-Alpha-)-Bisabolol, natural | Bisabolol | 0.10 |
| | (Symrise) | | |
| | Symrise perfume oil | Fragrance | 0.40 |

Heat part A and B separately to approx. 80° C. Add part B to part A, emulsify and cold-stir. Homogenise again at approx. 60° C. and add part C at approx. 35° C.

### EXAMPLE 2

### Skin-Browning Water-in-Oil Emulsion with UV-A/B Broad-Band Protection

| | Raw material name | | Content |
|---|---|---|---|
| Part | (manufacturer) | Chemical name | wt. % |
| A | Dehymuls PGPH ® (Cognis) | Polyglycerol-2 dipolyhydroxystearate | 3.0 |
| | Monomuls 90-O 18 ® (Cognis) | Glyceryl oleate | 1.0 |
| | Permulgin 2550 ® | Beeswax | 1.0 |
| | (Koster Keunen Holland) | | |
| | Myritol 318 ® (Cognis) | Caprylic/capric acid triglycerides | 6.0 |
| | Witconol TN ® (Witco) | C₁₂-C₁₅ alkyl benzoate | 6.0 |
| | Cetiol SN ® (Cognis) | Cetyl and stearyl isononanoate | 5.0 |
| | Copherol 1250 ® (Cognis) | Tocopherol acetate | 1.0 |
| | Solbrol P ® (Bayer) | Propylparaben | 0.1 |
| | Neo Heliopan ® AV (Symrise) | Ethylhexyl methoxycinnamate | 4.0 |
| | Neo Heliopan ® E 1000 | Isoamyl-p-methoxycinnamate | 4.0 |
| | (Symrise) | | |
| | Neo Heliopan ® MBC | 4-Methylbenzylidene camphor | 2.0 |
| | (Symrise) | | |
| | Neo Heliopan ® OS (Symrise) | Ethylhexyl salicylate | 3.0 |
| | Naringin | 4′,5,7-Trihydroxyflavone-7-O- | 0.50 |
| | | neohesperidoside | |
| | Octyl triazone | Ethylhexyl triazone | 1.0 |
| | Zinc oxide neutral (Symrise) | Zinc oxide | 7.0 |
| B | Water, dist. | Aqua (water) | 39.5 |
| | Trilon BD ® (BASF) | Disodium EDTA | 0.1 |
| | Phenoxyethanol | | 0.7 |
| | Solbrol M (Bayer) | Methylparaben | 0.2 |
| | Glycerine 99% | | 4.0 |
| | Neo Heliopan ® AP (Symrise), | Disodium phenyl dibenzimidazole | 10.0 |
| | 15% as sodium salt | tetrasulfonate | |
| | Benzophenone-4 | Benzophenone-4 | 0.5 |
| C | Perfume oil | Perfume | 0.3 |
| | Bisabolol | Bisabolol | 0.1 |

For part A all substances apart from the zinc oxide were heated to 85° C. and the zinc oxide was carefully dispersed in the mixture. The components of part B were mixed together, heated to 85° C. and added to part A whilst stirring. Part C was added to the mixture of parts A and B and the mixture was then homogenised with a dispersing tool.

### EXAMPLE 3

### Intensive Skin-Browning Oil-in-Water Emulsion with UVA/B Broad-Band protection

| | Raw material name | | Content |
|---|---|---|---|
| Part | (manufacturer) | Chemical name | wt. % |
| A | Arlacel 165 ® (ICI) | Glyceryl stearate and polyethylene glycol | 3.0 |
| | | 100-stearate | |
| | Emulgin B2 ® (Cognis) | Ceteareth-20 | 1.0 |
| | Lanette O ® (Cognis) | Cetyl and stearyl alcohol | 1.15 |
| | Myritol 318 ® (Cognis) | Caprylic/capric acid triglycerides | 5.0 |
| | Cetiol SN ® (Cognis) | Cetyl and stearyl isononanoate | 4.0 |
| | Abil 100 ® (Goldschmidt) | Dimethicone | 1.0 |
| | Bentone Gel MIO ® (Rheox) | Mineral oil and quaternium-18-hectorite | 3.0 |
| | | and propylene carbonate | |
| | Cutina CBS ® (Cognis) | Glyceryl stearate and cetyl alcohol and | 2.0 |
| | | stearyl alcohol and cetyl palmitate and | |
| | | cocoglyceride | |
| | Neo Heliopan ® 303 (Symrise) | Octocrylene | 7.0 |
| | Neo Heliopan ® BB (Symrise) | Benzophenone-3 | 1.0 |
| | Neo Heliopan ® MA (Symrise) | Menthyl anthranilate | 3.0 |
| | N,N-Dimethyl-4-aminobenzoic | | 3.0 |
| | acid-2-ethylhexyl ester | | |
| | Naringin | 4′,5,7-Trihydroxyflavone-7-O- | 2.0 |
| | | neohesperidoside | |
| | Titanium dioxide, microfine | Titanium dioxide | 5.0 |
| B | Water, dist. | | 50.15 |
| | Trilon BD ® (BASF) | Disodium EDTA | 0.1 |
| | Veegum ultra ® (Vanderbilt) | Magnesium aluminium sulfate | 1.0 |
| | Magnesium chloride | | 1.0 |
| | Natrosol 250 HHR (Aqualon) | Hydroxymethylcellulose | 0.3 |
| | L-Tyrosine ethyl ester | | 2.0 |
| | Cuivridone (Erbsloh) | Copper pyrrolidinate | 0.5 |
| | Glycerine | Glycerine | 3.0 |
| | Phenopip ® | Phenoxyethanol (and) methylparaben | 0.3 |
| | (Nipa Laboratorien) | (and) ethylparaben (and) butylparaben | |
| | | (and) propylparaben (and) isobutylparaben | |
| C | Perfume oil | | 0.3 |

For part A all substances apart from the titanium dioxide were heated to 85° C.; the titanium dioxide was carefully dispersed into the mixture. For part B all substances apart from the Veegum and Natrosol were mixed together, heated to 90° C., the Natrosol and Veegum dispersed into the mixture and the mixture added to part A whilst stirring. Part C was added to the mixture of parts A and B and the mixture was then homogenised with a dispersing tool.

### EXAMPLE 4

### Skin-Browning Oil-in-Water Emulsion with UVA/B Broad-Band Protection

| | Raw material name | | |
|---|---|---|---|
| Part | (manufacturer) | Chemical name | Content wt. % |
| A | Hostacerin DGMS ® (Clariant) | Polglyceryl-2-stearate | 3.0 |
| | Lanette 16 ® (Cognis) | Cetyl alcohol | 2.0 |
| | Prisorine 3505 ® (UniQema) | Isostearic acid | 0.5 |
| | Tegosoft TN ® (Goldschmidt) | C₁₂-C₁₅ alkyl benzoate | 2.0 |
| | Copherol 1250 ® (Cognis) | Tocopherol acetate | 0.5 |
| | Neutral oil (Symrise) | Caprylic/capric acid triglyceride | 5.0 |
| | Solbrol P ® (Bayer) | Propylparaben | 0.1 |
| | SF1214 ® (Bayer) | Cyclopentasiloxane, dimethicone | 1.0 |
| | Corapan TQ ® (Symrise) | Diethylhexyl-1,6-naphthalate | 3.0 |
| | Neo Heliopan ® HMS (Symrise) | Homosalate | 9.5 |
| | Neo Heliopan ® 357 (Symrise) | Butyl methoxy dibenzoylmethane | 0.6 |
| | Hesperidin | 3′,5,7-Trihydroxy-4′-methoxyflavone-7- | 0.2 |
| | | O-rutinoside | |
| | Keltrol T ® (Kelco) | Xanthan gum | 0.2 |
| B | Water, dist. | Aqua (water) | 48.75 |
| | Lanette E (Cognis) | Sodium cetearyl sulfate | 0.75 |
| | Glycerine 99% | Glycerine | 4.0 |
| | Phenoxyethanol (Symrise) | Phenoxyethanol | 0.7 |
| | Edeta BD ® (BASF) | Disodium EDTA | 0.1 |
| | Neo Heliopan ® Hydro (15% | Phenylbenzimidazole sulfonic acid | 6.7 |
| | aqueous solution neutralised with | | |
| | NaOH) (Symrise) | | |
| | Neo Heliopan ® AP (10% | Disodium phenyl dibenzimidazole | 10.0 |
| | aqueous solution neutralised with | tetrasulfonate | |
| | NaOH) | | |
| | (Symrise) | | |
| | Symdiol 68 (Symrise) | 1,2-Hexanediol, capryl glycol | 0.50 |
| | Solbrol M ® (Bayer) | Methylparaben | 0.2 |
| C | Symrise perfume oil (Symrise) | Perfume oil | 0.5 |
| | NaOH 10% aqueous solution | Sodium hydroxide | 0.2 |

Part A was heated to 80° C. After dissolving all constituents, the mixture was heated to 85° C., Keltrol added and the mixture stirred for 5 min. The mixture was then homogenised for 10 min with a dispersing tool. The mixture was heated to 85° C., part B added, the mixture stirred for 10 min at 80° C. and then homogenised at 60° C. Finally part C was added at room temperature and the mixture homogenised with a dispersing tool.

### EXAMPLE 5

### Skin-Browning O/W Cream

| | Raw material name | | |
|---|---|---|---|
| Part | (manufacturer) | INCI name | Content in wt. % |
| A | Dracorin GMS (Symrise) | Glyceryl stearate | 2.00 |
| | PCL-Solid (Symrise) | Stearyl heptanoate, stearyl caprylate | 2.00 |
| | Lanette O (Cognis) | Cetearyl alcohol | 3.00 |
| | PCL Liquid 100 (Symrise) | Cetearyl ethylhexanoate | 5.00 |
| | Isodragol (Symrise) | Triisononanoin | 2.00 |
| | Abil 350 (Degussa- | Dimethicone | 2.00 |
| | Goldschmidt) | | |
| | Dragoxat EH (Symrise) | Ethylhexyl ethylhexanoate | 3.00 |
| | Hesperidin | 3′,5,7-Trihydroxy-4′-methoxyflavone-7- | 0.30 |
| | | O-rutinoside | |
| B | Demineralised water | Water (aqua) | 69.05 |
| | Carbopol Ultrez-10 | Carbomer | 0.10 |
| | (Noveon) | | |
| | Keltrol RD (CP-Kelco) | Xanthan gum | 0.10 |
| | Emulsiphos (Symrise) | Potassium cetyl phosphate, hydrogenated | 2.00 |
| | | palm glycerides | |
| | Dragocid Liquid (Symrise) | Phenoxyethanol, methylparaben, | 0.80 |
| | | ethylparaben, butylparaben, | |
| | | propylparaben, isobutylparaben | |
| | Extrapone Camomile GW | Glycerine, water (aqua), Chamomilla | 0.50 |
| | (Symrise) | recutita (matricaria) flower extract | |
| | Extrapone Rosemary GW | Glycerine, water (aqua), Rosmarinus | 0.30 |
| | (Symrise) | officinalis (rosemary) leaf extract | |
| | Extrapone Green Tea GW | Glycerine, water (aqua), Camellia | 0.20 |
| | (Symrise) | sinensis leaf extract | |
| | Drago-Beta-Glucan | Water (aqua), butylene glycol, glycerine, | 0.30 |
| | (Symrise) | Avena sativa (oat) kernel extract | |
| | Propylene glycol-1,2 99P | Propylene glycol | 5.00 |
| | GC | | |
| | Glycerine 85 P. | Glycerine | 2.00 |
| C | Sodium hydroxide (10% | Sodium hydroxide | 0.25 |
| | aqueous solution) | | |
| D | Symrise perfume oil | Fragrance | 0.30 |

Pre-swell Carbopol Ultrez-10 and Keltrol RD in water and add the remaining raw materials from part B. Heat part A and B separately to approx. 80° C. Add part A to part B and emulsify whilst adding part C. Cold-stir and add part D at around 35° C. The pH of the end product should be around 5.5.

### EXAMPLE 6

### Skin-Browning Aerosol Foam with UV-B/UV-A Protection

| | Raw material name | | Content |
|---|---|---|---|
| Part | (manufacturer) | Chemical name | wt. % |
| A | Emulsiphos (Symrise) | Cetyl phosphate, hydrogenated palm | 1.50 |
| | | glycerides | |
| | Cutina MD (Cognis) | Glyceryl stearate | 2.00 |
| | Lanette 16 (Cognis) | Cetyl alcohol | 0.50 |
| | Texapon N 70 (Cognis) | Sodium laureth sulfate | 0.10 |
| | Neutral oil (Symrise) | Caprylic/capric triglyceride | 2.00 |
| | Tegosoft TN (Degussa) | C12-15 Alkyl benzoate | 2.00 |
| | Copherol 1250 (Cognis) | Tocopheryl acetate | 0.50 |
| | Solbrol P (Bayer) | Propylparaben | 0.10 |
| | Edeta BD (BASF) | Disodium EDTA | 0.10 |
| | Neo Heliopan ® AV (Symrise) | Ethylhexyl methoxycinnamate | 6.00 |
| | Neo Heliopan ® MBC (Symrise) | 4-Methylbenzylidene camphor | 4.00 |
| | Neo Heliopan ® 357 (Symrise) | Butyl methoxy dibenzoylmethane | 1.50 |
| | Neohesperidin | 3′,5,7-Trihydroxy-4′-methoxyflavone-7- | 0.20 |
| | | O-rutinoside | |
| B | Demineralised water | Water (aqua) | 58.80 |
| | Glycerine 99% | Glycerine | 3.00 |
| | Solbrol M (Bayer) | Methylparaben | 0.20 |
| | Phenoxyethanol (Symrise) | Phenoxyethanol | 0.70 |
| | Carbopol ETD 2050 (Noveon) | Carbomer | 0.10 |
| C | Sodium hydroxide 10% aq. | Sodium hydroxide | 2.90 |
| A | Emulsiphos (Symrise) | Cetyl phosphate, hydrogenated palm | 1.50 |
| | | glycerides | |
| | Cutina MD (Cognis) | Glyceryl stearate | 2.00 |
| | Lanette 16 (Cognis) | Cetyl alcohol | 0.50 |
| | Texapon N 70 (Cognis) | Sodium laureth sulfate | 0.10 |
| | Neutral oil (Symrise) | Caprylic/capric triglyceride | 2.00 |
| | Tegosoft TN (Degussa) | C12-15 Alkyl benzoate | 2.00 |
| | Copherol 1250 (Cognis) | Tocopheryl acetate | 0.50 |
| | Solbrol P (Bayer) | Propylparaben | 0.10 |
| | Edeta BD (BASF) | Disodium EDTA | 0.10 |
| | Neo Heliopan ® AV (Symrise) | Ethylhexyl methoxycinnamate | 6.00 |
| | Neo Heliopan ® MBC (Symrise) | 4-Methylbenzylidene camphor | 4.00 |
| | Neo Heliopan ® 357 (Symrise) | Butyl methoxy dibenzoylmethane | 1.50 |
| | Neo Heliopan ® Hydro (15% | Phenylbenzimidazole sulfonic acid | 13.30 |
| | aqueous solution neutralised with | | |
| | NaOH) (Symrise) | | |
| D | Symrise perfume oil | Fragrance (perfume) | 0.40 |
| | Alpha-Bisabolol (Symrise) | Bisabolol | 0.10 |

Heat part A to 85° C. For part B disperse Carbopol evenly in water, then add all other raw materials for part B and heat to 85° C. Add part B to part A whilst stirring. Add part C directly to part A/B and leave to cool. Add part D to part A/B/C and introduce into aerosol containers. The pH of the end product should be around 7.5.

### EXAMPLE 7

### Shampoo with Skin and Hair Browning Properties

| | Raw material name | | Content in |
|---|---|---|---|
| Part | (manufacturer) | INCI name | wt. % |
| A | Genapol LRO liquid | Sodium laureth sulfate | 37.00 |
| | (Cognis) | | |
| | Naringin | 4′,5,7-Trihydroxyflavone- | 0.50 |
| | | 7-O-neohesperidoside | |
| | Dragoderm (Symrise) | Glycerine, Triticum | 2.00 |
| | | vulgare (wheat) | |
| | | gluten, water (aqua) | |
| B | Demineralised water | Water (aqua) | 31.10 |
| | Merquat 550 (Ondeo | Polyquaternium-7 | 0.50 |
| | Nalco) | | |
| C | Demineralised water | Water (aqua) | 20.00 |
| | Comperlan 100 (Cognis) | Cocamide MEA | 0.50 |
| D | Tego Betaine L7 uncons. | Cocamidopropyl | 6.00 |
| | (Degussa-Goldschmidt) | betaine | |
| | Citric acid 10% | Citric acid | 0.30 |
| | EDETA B powder | Tetrasodium EDTA | 0.10 |
| | (BASF) | | |
| | Sodium benzoate | Sodium benzoate | 0.50 |
| | Sodium chloride | Sodium chloride | 1.00 |
| | Symrise perfume oil | Fragrance | 0.50 |

Dissolve Naringin and Dragoderm in Genapol LRO. Pre-dissolve Merquat 550 and Dra in water and add. Dissolve part C whilst stirring and heating and allow to cool. Dissolve part C in part A/B. Add the raw materials from part D one at a time and stir. The pH of the end product should be around 5.0.

### EXAMPLE 8

### Skin and Hair Browning Hair Conditioner with UV-B/UV-A Protection

| | Raw material name | | Content |
|---|---|---|---|
| Part | (manufacturer) | INCI name | wt. % |
| A | Lanette O (Cognis) | Cetearyl alcohol | 2.50 |
| | Eumulgin B 2 (Cognis) | Ceteareth-20 | 0.70 |
| | Neo Heliopan 357 | Butyl methoxy | 0.50 |
| | | dibenzoylmethane | |
| | Neo Heliopan ® E 1000 | Isoamyl p-methoxycinnamate | 2.00 |
| | (Symrise) | | |
| | Naringin | 4′,5,7-Trihydroxyflavone-7-O- | 0.20 |
| | | neohesperidoside | |
| B | Demineralised water | Water (aqua) | 91.57 |
| | Crotein Q (Croda) | Hydroxypropyltrimonium | 1.00 |
| | | hydrolysed collagen | |
| | Dehyquart SP | Quaternium-52 | 0.50 |
| | Citric acid | Citric acid | 0.13 |
| | Symrise perfume oil | Fragrance (perfume) | 0.40 |
| C | Phenonip (Clariant) | Phenoxyethanol (and) | 0.50 |
| | | methylparaben (and) | |
| | | ethylparaben (and) | |
| | | butylparaben (and) | |
| | | propylparaben (and) | |
| | | isobutylparaben | |

Heat part A to 70° C. Dissolve the raw materials for part B with the exception of the perfume oil in water, heat to 90° C. and add this solution to part A whilst stirring. Allow the emulsion to cool to 40° C., stirring slowly, and add the perfume oil whilst stirring. After storing for 24 hours, add the Phenopip whilst stirring. The pH of the end product should be around 3.5.

### EXAMPLE 9

### Skin-Browning Moisture Cream O/W

| | Raw material name | | |
|---|---|---|---|
| Part | (manufacturer) | Chemical name | Content wt. % |
| A | PCL liquid (Symrise) | Cetearyl ethylhexanoate, isopropyl | 3.0 |
| | | myristate | |
| | Dragophos S (Symrise) | Sodium dihydroxycetyl phosphate | 2.0 |
| | Isodragol (Symrise) | Triisononanoin | 7.0 |
| | Dracorin GMS (Symrise) | Glyceryl stearate | 2.0 |
| | Lanette 18 (Care Chemicals) | Stearyl alcohol | 4.5 |
| | Pseudoceramide 391 (Symrise) | N-(1-Hexadecanoyl)-4-hydroxy-L-proline- | 0.5 |
| | | (1-hexadecyl) ester | |
| | Dow Corning 200 Fluid (Dow | Dimethicone | 2.0 |
| | Corning) | | |
| B | Water | Water (aqua) | 73.5 |
| | Hydrolite-5 (Symrise) | Pentylene glycol | 3.0 |
| | Naringin | 4′,5,7-Trihydroxyflavone-7-O- | 1.0 |
| | | neohesperidoside | |
| | Dragocid Liquid (Symrise) | Methylparaben, phenoxyethanol, | 0.8 |
| | | ethylparaben, butylparaben, | |
| | | propylparaben, isobutylparaben | |
| | Citric acid 10% solution | Citric acid | 0.35 |
| C | Perfume oil | Fragrance | 0.35 |

Swell Carbopol in water. Heat phases A and B separately to 80° C. Add phase B to phase A, and only then emulsify. Cold-stir with a paddle agitator. Reduce the stirring speed as the temperature falls. At 40° C. add the raw materials for phase C.

### EXAMPLE 10

### Skin-Browning Face Cream O/W

| | Raw material name | | |
|---|---|---|---|
| Part | (manufacturer) | Chemical name | Content wt. % |
| A | Dracorin 100 s.e. P (Symrise) | Glyceryl stearate, PEG-100 stearate | 8.0 |
| | Dracorin GMS (Symrise) | Glyceryl stearate | 3.0 |
| | Paraffin oil 5 grade E (Parafluid) | Paraffinum liquidum | 4.0 |
| | Lanette 16 (Care Chemicals) | Cetyl alcohol | 2.0 |
| | Isopropyl myristate (Symrise) | Isopropyl myristate | 8.0 |
| | Abil 350 (Goldschmidt) | Dimethicone | 0.3 |
| B | Water | Water (aqua) | 67.35 |
| | Propylene glycol-1,2 99 P GC | Propylene glycol | 5.0 |
| | (Dow Benelux) | | |
| | Hesperidin | 3′,5,7-Trihydroxy-4′-methoxyflavone-7- | 0.5 |
| | | O-rutinoside | |
| | Naringin | 4′,5,7-Trihydroxyflavone-7-O- | 0.5 |
| | | neohesperidoside | |
| | Neo-Dragocid powder (Symrise) | Methylparaben, sorbic acid, | 0.8 |
| | | dehydroacetic acid, propylparaben | |
| | Sodium hydroxide 10% solution | Sodium hydroxide | 0.25 |
| | Perfume oil | Fragrance | 0.30 |

Heat phase A and B separately to approx. 80° C. Add phase B to phase A in an Ultra-Turrax agitator and emulsify. Cold-stir the cream with a paddle agitator, reducing the speed from 250 rpm as the temperature falls. Add phase C at approx. 40° C.

### EXAMPLE 11

### Self-Tanning Cream O/W

| | | | Content |
|---|---|---|---|
| | Raw material name | | in |
| Part | (manufacturer) | INCI name | wt. % |
| A | Dracorin CE (Symrise) | Glyceryl stearate citrate | 5.00 |
| | Lanette 16 (Cognis) | Cetyl alcohol | 1.00 |
| | Isopropyl palmitate (Croda) | Isopropyl palmitate | 4.00 |
| | PCL Liquid (Symrise) | Cetearyl ethylhexanoate | 3.00 |
| | Dragoxat EH (Symrise) | Ethylhexyl ethylhexanoate | 3.00 |
| | Neutral oil | Caprylic/Capric triglyceride | 6.00 |
| | Naringin | 4′,5,7-Trihydroxyflavone- | 0.5 |
| | | 7-O-neohesperidoside | |
| | Abil 200 (Degussa- | Dimethicone | 0.50 |
| | Goldschmidt) | | |
| B | Demineralised water | Water (aqua) | 53.30 |
| | EDETA BD (BASF) | Disodium EDTA | 0.10 |
| | Keltrol T (Danby-Chemie) | Xanthan gum | 0.30 |
| | Glycerine 99.5% | Glycerine | 1.50 |
| | Hydrolite-5 (Symrise) | Pentylene glycol | 3.50 |
| C | Sepigel 305 | Polyacrylamide, C13-14 | 1.00 |
| | | isoparaffin, laureth-7 | |
| D | Demineralised water | Water (aqua) | 10.00 |
| | Dihydroxyacetone (Merck) | Dihydroxyacetone | 5.00 |
| | Ethanol 96% | Ethanol | 2.00 |
| E | Perfume oil (Symrise) | Fragrance | 0.30 |

Heat phase A and B separately to approx. 80° C. Add phase B to phase A without stirring and homogenise. Cool to 50° C. and add phase C whilst stirring and homogenise. Cool to 35° C. and add phase D. Add phase E at room temperature.

### EXAMPLE 12

### Cytotoxicity Determination

B16V mouse melanoma cells are disseminated in a 96-well microtitre plate in a concentration of 2×10⁴ cells/well (B16V). After cultivation for 24 h at 37° C. and 5% CO₂ in RPMI medium (B16V cells), enriched with 10% foetal calf serum, the medium is drawn off. Various concentrations of the test substances, dissolved in fresh medium enriched with 5% foetal calf serum, are added and incubated for a further 48 h. In parallel the cells are incubated with SDS as standard in concentrations of 0.01 mM, 0.1 mM, 1 mM and 10 mM. After incubation the medium is drawn off and the cells are incubated for 2 h with MTT (3-[4,5-dimethylthiazol-2-yl]2,5-diphenyl tetrazolium bromide). After extraction of the dye (MTT) with acetic acid SDS in DMSO (10 min), the absorption (A) is measured at 570 nm.

The mean and standard deviation of the controls, blanks and samples are calculated. The mean of the blank is subtracted from the means of the controls and samples. The viability of the cells is stated as a percentage relative to the controls (100%):

Viability (%)=[(A_{test compound}/A_{control})×100]

The IC₅₀ (mean inhibitory concentration) indicates the test substance concentration at which 50% of the cells are vital.

TABLE 1

| Test substance | IC₅₀ (mM) |
|---|---|
| Apigenin | 0.033 |
| Quercetin | 0.048 |
| Kaempferol | 0.043 |
| Luteolin | 0.037 |
| Neohesperidin | >4.0 |
| Hesperidin | 1.500 |
| Naringin | >10.0 |
| Hesperetin | 0.040 |
| Naringenin | 0.030 |

As can be seen from Table 1, neohesperidin, hesperidin and in particular naringin are extremely cell-compatible (IC50, MTT >1 mM), whereas all other flavonoids have a cytotoxic effect even at low concentrations (IC50, MTT <0.05 mM).

### EXAMPLE 13

### Pigmenting Effect

B16V mouse melanoma cells are disseminated in a 96-well microtitre plate in a concentration of 5×10³ cells/well. After cultivation for 24 h at 37° C. and 5% CO₂ in RPMI medium, enriched with 10% foetal calf serum, various concentrations of the test substances and 10 nM α-MSH (α-melanocyte stimulating hormone) are added and incubated for a further 96 h. The maximum concentration of the test substances used corresponds to 0.1 times the value of the IC₂₀ value of the cytotoxicity assay (=non-cytotoxic concentration range). After incubation, SDS and NaOH (final concentrations: 1 mM and 1 M respectively) are added to the culture medium and the absorption (A) is measured after 3 h at 400 nm.

The stimulation of pigmentation in the presence of the test compounds was calculated using the following equation:

Stimulation of pigmentation (%)=[(A test compound/A control)×100]−100

From the stimulation of pigmentation (%) in a series of dilutions of test compounds, the EC₅₀ for each test compound was calculated. This is the concentration of a test compound at which pigmentation is stimulated by 50%.

TABLE 2

| Test substance | EC₅₀ (mM) |
|---|---|
| Apigenin | inactive |
| Quercetin | 0.015 |
| Kaempferol | inactive |
| Luteolin | inactive |
| Neohesperidin | 0.0010 |
| Hesperidin | 0.0015 |
| Naringin | 0.0005 |
| Hesperetin | inactive |
| Naringenin | inactive |

Table 2 shows that neohesperidin, hesperidin and in particular naringin are highly effective (EC50, melanin induction <0.002 mM). Quercetin also has a good effect, but the effective concentration is very close to the cytotoxic range (see example 14, table 3), as a result of which product safety in terms of cytotoxicity cannot be guaranteed.

### EXAMPLE 14

### Calculation of the Safety Factor SF

The so-called safety factor SF is used to estimate product safety in terms of cytotoxicity. This is calculated as follows:

Safety factor SF=(IC50 cytotox)/(EC50 pigmenting effect)

The SF indicates the factor by which the amount necessary for pigmentation can be exceeded without the onset of a cytotoxic effect.

TABLE 3

| | IC₅₀ cytotox | EC₅₀ pigm. | |
|---|---|---|---|
| Test substance | (mM) | effect (mM) | SF |
| Apigenin | 0.033 | inactive | n.c. |
| Quercetin | 0.048 | 0.015 | 3.2 |
| Kaempferol | 0.043 | inactive | n.c. |
| Luteolin | 0.037 | inactive | n.c. |
| Neohesperidin | >4.0 | 0.0010 | 4000 |
| Hesperidin | 1.500 | 0.0015 | 1000 |
| Naringin | >10.0 | 0.0005 | 20000 |
| Hesperetin | 0.040 | inactive | n.c. |
| Naringenin | 0.030 | inactive | n.c. |

n.c. not calculable

To guarantee product safety in terms of cytotoxicity in the case of quercetin, the concentration used in vivo according to the above definition must not exceed 3.2 times the amount needed for browning. By contrast, the usage concentration of naringin, hesperidin and neohesperidin can be 20,000 times, 1000 times and 4000 times respectively the amount needed for browning.

1. A method for the browning of skin or hair in vivo comprising contacting said skin or hair with a preparation comprising a compound having formula (I) as an agent 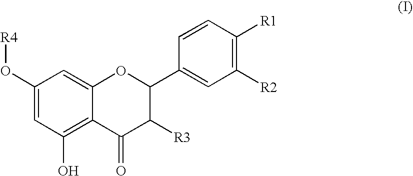
wherein:
R1 and R2 are mutually independently H, OH, C₁-C₁₀-alkyl, C₁-C₁₀—O-alkyl or O-prenyl,
R3 is H, OH, O-glucose or O-rhamnose
and
R4 is a monosaccharide radical or an oligosaccharide radical having 2, 3, 4 or 5 carbohydrate units,
with the proviso that the compound having formula (I) is not used in the form of a preparation based on Citrus aurantium dulcis.

2. A method according to claim 1, wherein the compound having formula (I) is used in an amount which is in the range between the minimum necessary amount for browning in vivo and 1000 times the amount for browning.

3. A formulation for the browning of skin or hair, comprising
at least one compound having formula (I), 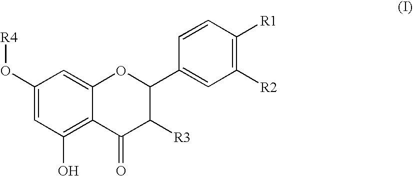
wherein:
R1 and R2 are mutually independently H, OH, C₁-C₁₀-alkyl, C₁-C₁₀—O-alkyl or O-prenyl,
R3 is H, OH, O-glucose or O-rhamnose
and
R4 is a monosaccharide radical or an oligosaccharide radical having 2, 3, 4 or 5 carbohydrate units, in an amount which is in the range between the minimum necessary amount for browning in vivo and 1000 times said amount, with the proviso that the compound having formula (I) is not used in the form of a preparation based on Citrus aurantium dulcis,
a conditioning or cleansing substance for skin or hair.

4. A method according to claim 1, wherein in the compound having formula (I):
R1 and R2 are mutually independently H, OH, OMe or CH₃
and
R3 is H
and
R4 is a monosaccharide radical or an oligosaccharide radical having 2, 3, 4 or 5 carbohydrate units.

5. A method according to claim 1, wherein in the compound having formula (I):
R1 and R2 are mutually independently H, OH or OMe
and
R3 is H
and
R4 is (i) a monosaccharide radical, preferably selected from the group consisting of glucose, galactose, rhamnose, xylose and glucuronic acid, or (ii) a disaccharide radical, whose sugar units are the same or different and are preferably selected from the group consisting of glucose, galactose, rhamnose, xylose and glucuronic acid.

6. A method according to claim 1, wherein the compound having formula (I) is naringin, hesperidin or neohesperidin.

7. A process for the browning of skin or hair, comprising:
applying to said skin or hair in vivo an effective amount of a compound having formula (I), 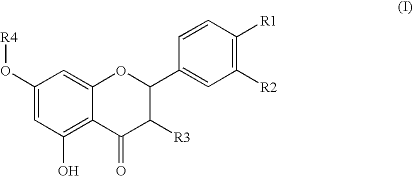
wherein:
R1 and R2 are mutually independently H, OH, C₁-C₁₀-alkyl, C₁-C₁₀—O-alkyl or O-prenyl,
R3 is H, OH, O-glucose or O-rhamnose
and
R4 is a monosaccharide radical or an oligosaccharide radical having 2, 3, 4 or 5 carbohydrate units, in an amount which is in the range between the minimum necessary amount for browning in vivo and 1000 times said amount, with the proviso that the compound having formula (I) is not used in the form of a preparation based on Citrus aurantium dulcis.
